(19) United States
(12) Patent Application Publication
(10) Pub. No.: US 2017/0342064 A1
(43) Pub. Date: Nov. 30, 2017

(54) COMPOUNDS FOR THE TREATMENT OF CANCER

(71) Applicant: Bayer Pharma Aktiengesellschaft, Berlin (DE)

(72) Inventors: Volker SCHULZE, Hohen Neuendorf, OT Bergf (DE); Hans-Georg LERCHEN, Leverkusen (DE); Ulrich LÜCKING, Berlin (DE); Antje Margret WENGNER, Berlin (DE); Gerhard SIEMEISTER, Berlin (DE); Philip LIENAU, Berlin (DE); Ursula KRENZ, Leichlingen (DE)

(73) Assignee: Bayer Pharma Aktiengesellschaft, Berlin (DE)

(21) Appl. No.: 15/534,407

(22) PCT Filed: Dec. 7, 2015
(86) PCT No.: PCT/EP2015/078871

§ 371 Date: Jun. 8, 2017

(30) Foreign Application Priority Data
Dec. 9, 2014 (EP) 14196859.4

(51) Int. Cl. C07D 471/04

(52) U.S. Cl. CPC C07D 471/04

(57) ABSTRACT

The present invention relates to prodrug derivatives of Mps-1 kinase inhibitors, and their use for the treatment and/or prophylaxis of diseases.

The present invention relates to prodrug derivatives of Mps-1 kinase inhibitors, and their use for the treatment and/or prophylaxis of diseases.

### BACKGROUND OF THE INVENTION

Mps-1 (Monopolar Spindle 1) kinase (also known as Tyrosine Threonine Kinase, UK) is a dual specificity Ser/Thr kinase which plays a key role in the activation of the mitotic checkpoint (also known as spindle checkpoint, spindle assembly checkpoint) thereby ensuring proper chromosome segregation during mitosis [Abrieu A et al., Cell, 2001, 106, 83-93]. Every dividing cell has to ensure equal separation of the replicated chromosomes into the two daughter cells. Upon entry into mitosis, chromosomes are attached at their kinetochores to the microtubules of the spindle apparatus. The mitotic checkpoint is a surveillance mechanism that is active as long as unattached kinetochores are present and prevents mitotic cells from entering anaphase and thereby completing cell division with unattached chromosomes [Suijkerbuijk S J and Kops G J, Biochemica et Biophysica Acta, 2008, 1786, 24-31; Musacchio A and Salmon E D, Nat Rev Mol Cell Biol., 2007, 8, 379-93]. Once all kinetochores are attached in a correct amphitelic, i.e. bipolar, fashion with the mitotic spindle, the checkpoint is satisfied and the cell enters anaphase and proceeds through mitosis. The mitotic checkpoint consists of a complex network of a number of essential proteins, including members of the MAD (mitotic arrest deficient, MAD 1-3) and Bub (Budding uninhibited by benzimidazole, Bub 1-3) families, the motor protein CENP-E, Mps-1 kinase as well as other components, many of these being over-expressed in proliferating cells (e.g. cancer cells) and tissues [Yuan B et al., Clinical Cancer Research, 2006, 12, 405-10]. The essential role of Mps-1 kinase activity in mitotic checkpoint signalling has been shown by shRNA-silencing, chemical genetics as well as chemical inhibitors of Mps-1 kinase [Jelluma N et al., PLos ONE, 2008, 3, e2415; Jones M H et al., Current Biology, 2005, 15, 160-65; Dorer R K et al., Current Biology, 2005, 15, 1070-76; Schmidt M et al., EMBO Reports, 2005, 6, 866-72].

There is ample evidence linking reduced but incomplete mitotic checkpoint function with aneuploidy and tumorigenesis [Weaver B A and Cleveland D W, Cancer Research, 2007, 67, 10103-5; King R W, Biochimica et Biophysica Acta, 2008, 1786, 4-14]. In contrast, complete inhibition of the mitotic checkpoint has been recognised to result in severe chromosome missegregation and induction of apoptosis in tumour cells [Kops G J et al., Nature Reviews Cancer, 2005, 5, 773-85; Schmidt M and Medema R H, Cell Cycle, 2006, 5, 159-63; Schmidt M and Bastians H, Drug Resistance Updates, 2007, 10, 162-81].

Therefore, mitotic checkpoint abrogation through pharmacological inhibition of Mps-1 kinase or other components of the mitotic checkpoint represents a new approach for the treatment of proliferative disorders including solid tumours such as carcinomas and sarcomas and leukaemias and lymphoid malignancies or other disorders associated with uncontrolled cellular proliferation.

Different compounds have been disclosed in prior art which show an inhibitory effect on Mps-1 kinase: WO 2009/024824 A1 discloses 2-Anilinopurin-8-ones as inhibitors of Mps-1 for the treatment of proliferate disorders. WO 2010/124826 Al discloses substituted imidazoquinoxaline compounds as inhibitors of Mps-1 kinase. WO 2011/026579 A1 discloses substituted aminoquinoxalines as Mps-1 inhibitors. WO 2011/064328 A1, WO 2011/063907 A1, WO 2011/063908 A1, and

WO 2012/143329 A1 relate to [1,2,4]-triazolo-[1,5-α]-pyridines and their use for inhibition of Mps-1 kinase.

The above mentioned patent applications which are related to [1,2,4]-triazolo-[1,5-α]-pyridines mainly focus on the effectiveness of the compounds in inhibiting Mps-1 kinase, expressed by the half maximal inhibitory concentration (IC₅₀) of the compounds.

In addition, as one of ordinary skill in the art knows, there a many more factors determining the druglikeness of a compound. The objective of a pre-clinical development is to assess e.g. safety, toxicity, pharmacokinetics and metabolism parameters prior to human clinical trials. One important factor for assessing the druglikeness of a compound is the metabolic stability. The metabolic stability of a compound can be determined e.g. by incubating the compound with a suspension of liver microsomes from e.g. a rat, a dog and/or a human (for details see experimental section).

Another important factor for assessing the druglikeness of a compound for the treatment of cancer is the inhibition of cell proliferation which can be determined e.g. in a HeLa cell proliferation assay (for details see experimental section).

The successful delivery of a pharmaceutical to a patient is of critical importance in the treatment of disorders as well. The use of many clinical drugs with known bioactive properties is limited by the drugs' very low water solubility, making for example intravenous administration of the active ingredient difficult.

Intravenous (i.v.) medication administration refers to the process of giving medication directly into a patient's vein. Methods of administering i.v. medication may include giving the medication by rapid injection (push) into the vein using a syringe, giving the medication intermittently over a specific amount of time using an i.v. secondary line, or giving the medication continuously mixed in the main i.v. solution.

The primary purpose of giving i.v. medications is to initiate a rapid systemic response to medication. It is one of the fastest ways to deliver medication. The drug is immediately available to the body. It is easier to control the actual amount of drug delivered to the body by using the i.v. method and it is also easier to maintain drug levels in the blood for therapeutic response.

As a result of low water solubility, many drugs often are formulated in co-solvent pharmaceutical vehicles or as prodrugs.

A prodrug is an active drug chemically transformed into a derivative which by virtue of chemical or enzymatic attack is converted to the parent drug within the body before or after reaching the site of action. The process of converting an active drug into inactive form is called drug latentiation. Prodrugs can be carrier-linked-prodrugs and bioprecursors. The carrier-linked prodrug results from a temporary linkage of the active molecule with a transport moiety. Such prodrugs are less active or inactive compared to the parent active drug. The transport moiety will be chosen for its non-toxicity and its ability to ensure the release of the active principle with efficient kinetics. Whereas the bioprecursors result from a molecular modification of the active principle itself by generation of a new molecule that is capable of being a substrate to the metabolizing enzymes releasing the active principle as a metabolite.

Prodrugs are prepared to alter the drug pharmacokinetics, improve stability and solubility, decrease toxicity, increase specificity, and/or increase duration of the pharmacological effect of the drug. By altering pharmacokinetics the drug bioavailability is increased by increasing absorption, distribution, biotransformation, and/or excretion of the drug.

In designing the prodrugs, it is important to consider the following factors: a) the linkage between the carrier and the drug is usually a covalent bond, b) the prodrug is inactive or less active than the active principle, c) the prodrug synthesis should not be expensive, d) the prodrug has to be reversible or bioreversible derivative of the drug, and e) the carrier moiety must be non-toxic and inactive when released.

Prodrugs are usually prepared by: a) formation of ester, hemiesters, carbonate esters, nitrate esters, amides, hydroxamic acids, carbamates, imines, mannich bases, and enamines of the active drug, b) functionalizing the drug with azo, glycoside, peptide, and ether functional groups, c) use of polymers, salts, complexes, phosphoramides, acetals, hemiacetals, and ketal forms of the drug (for example, see Andrejus Korolkovas's, “Essentials of Medicinal Chemistry”, pp. 97-118).

It is therefore an object of the present invention to identify an Mps-1 kinase inhibiting compound or a prodrug derivative thereof which is characterized by a high druglikeness and which can be administered intravenously.

### SUMMARY OF THE INVENTION

The present invention relates to compounds of general formula (I) :

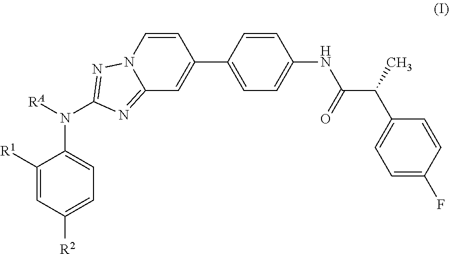

in which:
R^{A} represents —C(═O)—O—C(R⁴)(R⁵)—O—C(═O)—C(R³)(NH₂)—R⁶;
R¹ represents a group selected from methoxy- and 2,2,2-trifluoroethoxy-;
R² represents a group selected from:

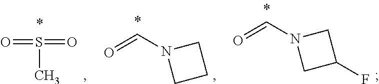

wherein “*” indicates the point of attachment to the phenyl ring R² is attached to;
R³ represents a hydrogen atom or a methyl-group;
R⁴ and R⁵, independently from each other, represent a hydrogen atom or a C₁-C₃-alkyl-group;
R⁶ represents a hydrogen atom or a C₁-C6-alkyl-group;
   or an N-oxide, a hydrate, a solvate, or a salt thereof, or a mixture of same.

### DETAILED DESCRIPTION OF THE INVENTION

The terms as mentioned in the present text have the following meanings :

The term “halogen atom” or “halo-” is to be understood as meaning a fluorine, chlorine, bromine or iodine atom.

The term “C₁-C₆-alkyl-” is to be understood as meaning a linear or branched, saturated hydrocarbon group having 1, 2, 3, 4, 5 or 6 carbon atoms, e.g. a methyl-, ethyl-, propyl-, butyl-, pentyl-, hexyl-, iso-propyl-, iso-butyl-, sec-butyl-, tert-butyl-, iso-pentyl-, 2-methylbutyl-, 1-methylbutyl-, 1-ethylpropyl-, 1,2-dinnethylpropyl-, neo-pentyl-, 1,1-dinnethylpropyl-, 4-methylpentyl-, 3-methylpentyl-, 2-methylpentyl-, 1-methylpentyl-, 2-ethylbutyl-, 1-ethylbutyl-, 3,3-dimethylbutyl-, 2,2-dimethylbutyl-, 1,1-dimethylbutyl-, 2,3-dimethylbutyl-, 1,3-dimethylbutyl-, or 1,2-dimethylbutyl-group, or an isomer thereof. Particularly, said group has 1, 2, 3 or 4 carbon atoms (“C₁-C₄-alkyl-”), e.g. a methyl-, ethyl-, propyl-, butyl-, iso-propyl-, iso-butyl-, sec-butyl-, tert-butyl-group, more particularly 1, 2 or 3 carbon atoms (“C₁-C₃-alkyl-”), e.g. a methyl-, ethyl-, n-propyl- or iso-propyl-group.

As used herein, the term “leaving group” refers to an atom or a group of atoms that is displaced in a chemical reaction as stable species taking with it the bonding electrons. Preferably, a leaving group is selected from the group comprising: halo, in particular chloro, bromo or iodo, methanesulfonyloxy-, p-toluenesulfonyloxy-, trifluoromethanesulfonyloxy-, nonafluorobutanesulfonyloxy-, (4-bromo-benzene)sulfonyloxy-, (4-nitro-benzene)sulfonyloxy-, (2-nitro-benzene)-sulfonyloxy-, (4-isopropyl-benzene)sulfonyloxy-, (2,4,6-tri-isopropyl-benzene)-sulfonyloxy-, (2,4,6-trimethyl-benzene)sulfonyloxy-, (4-tertbutyl-benzene)sulfonyloxy-, benzenesulfonyloxy-, and (4-methoxy-benzene)sulfonyloxy-.

As used herein, the term “PG” refers to a protecting group for hydroxy groups e.g. a TMS group or TBDPS group as decribed for example in T. W. Greene and P.G.M. Wuts in Protective Groups in Organic Synthesis, 3^{rd} edition, Wiley 1999 (TMS=trimethylsilyl, TBDPS=tert-butyldiphenylsilyl).

As used herein, the term “PG²” refers to a protecting group for amino groups e.g. a Boc group as descibed for example in T.W. Greene and P.G.M. Wuts in Protective Groups in Organic Synthesis, 3^{rd} edition, Wiley 1999 (Boc=tert-butyloxycarbonyl).

The present invention relates to compounds of general formula (I):

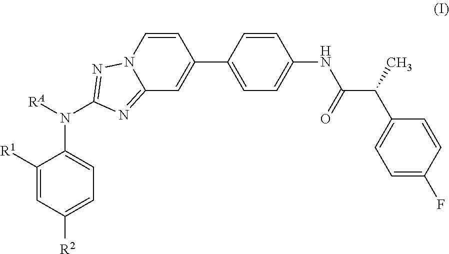

in which:
R^{A} represents —C(═O)—O—C(R⁴)(R⁵)—O—C(═O)—C(R³)(NH₂)—R⁶;
R¹ represents a group selected from methoxy- and 2,2,2-trifluoroethoxy-;
R² represents a group selected from:

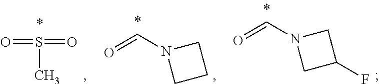

wherein “*” indicates the point of attachment to the phenyl ring R² is attached to;
R³ represents a hydrogen atom or a methyl-group;
R⁴ and R⁵, independently from each other, represent a hydrogen atom or a C₁-C₃-alkyl-group,
R⁶ represents a hydrogen atom or a C₁-C₆-alkyl-group;
or an N-oxide, a hydrate, a solvate, or a salt thereof, or a mixture of same.
R^{A} represents R⁶—C(R³)(NH₂)—C(═O)—O—C(R⁴)(R⁵)—O—C(═O)—.

In a preferred embodiment, R^{A} represents a group selected from:

R⁶—C(R³)(NH₂)—C(═O)—O—CH₂—O—C(═O)—,

R⁶—C(R³)(NH₂)—C(═O)—O—C(H)(CH₃)—O—C(═O)—, and

R⁶—C(R³)(NH₂)—C(═O)—O—C(H)(C(H)(CH₃)₂)—O—C(═O)—.

In another preferred embodiment, R^{A} represents a group selected from:

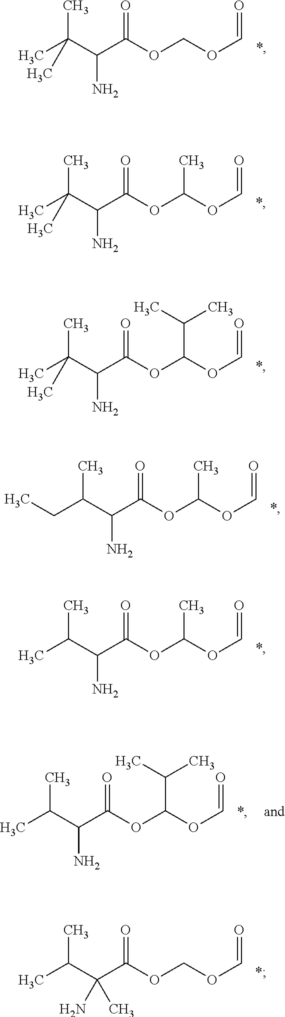

wherein “*” indicates the point of attachment to the nitrogen atom R^{A} is attached to.

In another preferred embodiment, R^{A} represents a group selected from:

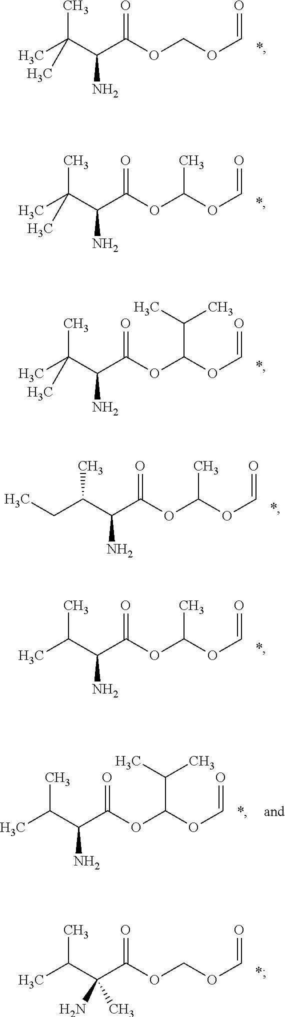

wherein “*” indicates the point of attachment to the nitrogen atom R^{A} is attached to.

In another preferred embodiment, R^{A} represents a group selected from:

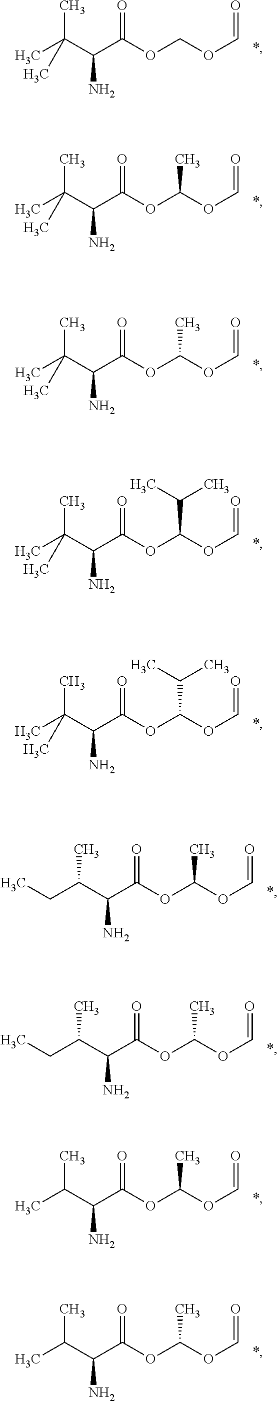

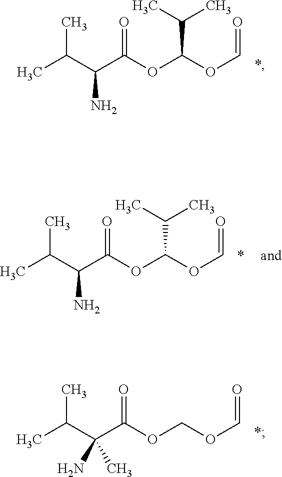

wherein “*” indicates the point of attachment to the nitrogen atom R^{A} is attached to.

R¹ represents a group selected from methoxy- and 2,2,2-trifluoroethoxy-.

In a preferred embodiment, R¹ represents a 2,2,2-trifluoroethoxy-group.

In another preferred embodiment, R¹ represents a methoxy-group.

R² represents a group selected from:

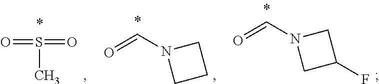

wherein “*” indicates the point of attachment to the phenyl ring R² is attached to.

In a preferred embodiment, R² represents a group selected from

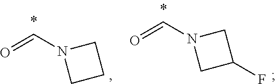

wherein “*” indicates the point of attachment to the phenyl ring R² is attached to.

In another preferred embodiment, R² represents

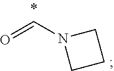

wherein “*” indicates the point of attachment to the phenyl ring R² is attached to.

In another preferred embodiment, R² represents

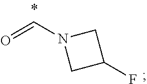

wherein “*” indicates the point of attachment to the phenyl ring R² is attached to.

In another preferred embodiment, R² represents a —S(═O)₂CH₃ group.

R³ represents a hydrogen atom or a methyl-group.

In a preferred embodiment, R³ represents a hydrogen atom.

In another preferred embodiment, R³ represents a methyl-group.

R⁴ and R⁵, independently from each other, represent a hydrogen atom or a C₁-C₃-alkyl-group.

In a preferred embodiment, R⁴ and R⁵, independently from each other, represent a hydrogen atom or a methyl- or an iso-propyl-group.

In another preferred embodiment, R⁴ represents a hydrogen atom or a C₁-C₃-alkyl-group, and R⁵ represents a hydrogen atom.

In another preferred embodiment, R⁴ represents a hydrogen atom or a methyl- or iso-propyl-group, and R⁵ represents a hydrogen atom.

In another preferred embodiment, R⁴ and R⁵ each represent a hydrogen atom.

In another preferred embodiment, R⁴ represents a methyl-group, and R⁵ represents a hydrogen atom.

In another preferred embodiment, R⁴ represents an iso-propyl-group, and R⁵ represents a hydrogen atom.

In another preferred embodiment, R⁴ represents a hydrogen atom or a C₁-C₃-alkyl-group.

In another preferred embodiment, R⁴ represents a hydrogen atom or a methyl-group.

In another preferred embodiment, R⁴ represents a hydrogen atom.

In another preferred embodiment, R⁴ represents a methyl-group.

In another preferred embodiment, R⁵ represents a hydrogen atom.

R⁶ represents a hydrogen atom or a C₁-C₆-alkyl-group.

In a preferred embodiment, R⁶ represents a C₁-C₆-alkyl-group.

In another preferred embodiment, R⁶ represents a hydrogen atom or a C₁-C₄-alkyl-group.

In another preferred embodiment, R⁶ represents a C₁-C₄-alkyl-group.

In another preferred embodiment, R⁶ represents a group selected from: iso-propyl, tert-butyl, and H₃C—CH₂—C(H)(CH₃)—.

In a further embodiment of the above-mentioned aspect, the invention relates to compounds of formula (I), according to any of the above-mentioned embodiments, in the form of an N-oxide, a hydrate, a solvate, or a salt thereof, or a mixture of same.

It is to be understood that the present invention relates also to any combination of the preferred embodiments described above.

Some examples of combinations are given hereinafter. However, the invention is not limited to these combinations.

In a preferred embodiment, the invention relates to compounds of the formula (I), supra, in which:

R^{A} represents a group selected from:

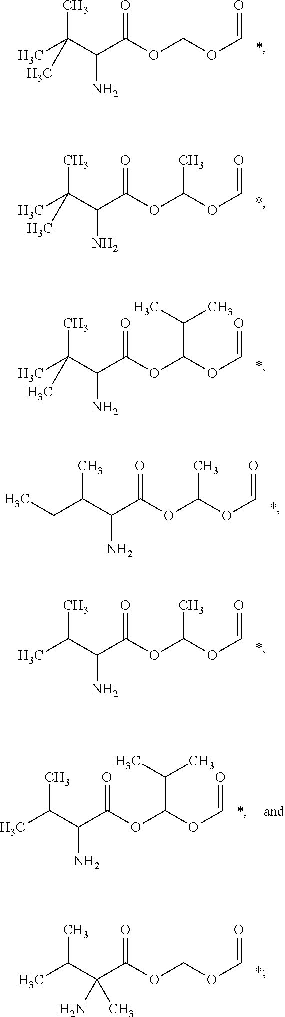

wherein “*” indicates the point of attachment to the nitrogen atom R^{A} is attached to;

R¹ represents a group selected from methoxy- and 2,2,2-trifluoroethoxy-; and

R² represents a group selected from:

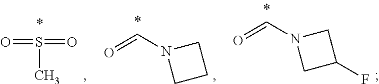

wherein “*” indicates the point of attachment to the phenyl ring R² is attached to;
   or an N-oxide, a hydrate, a solvate, or a salt thereof, or a mixture of same.

In another preferred embodiment, the invention relates to compounds of the formula (Ia), (Ib), (Ic), (Id), (Ie) or (If):

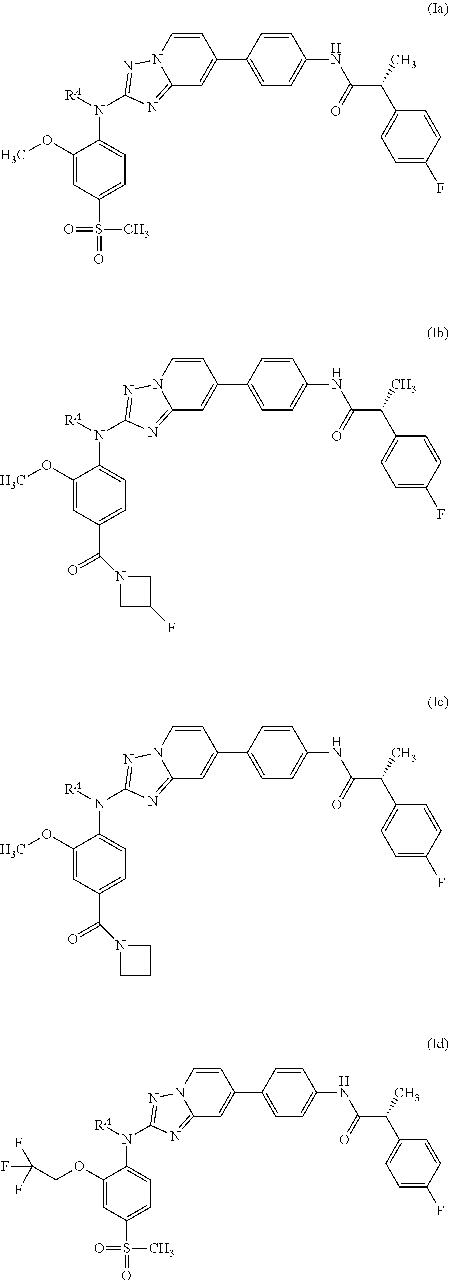

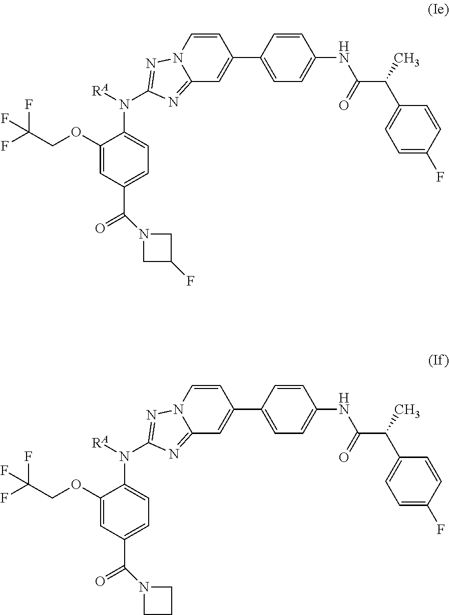

in which:

R^{A} represents a group selected from:

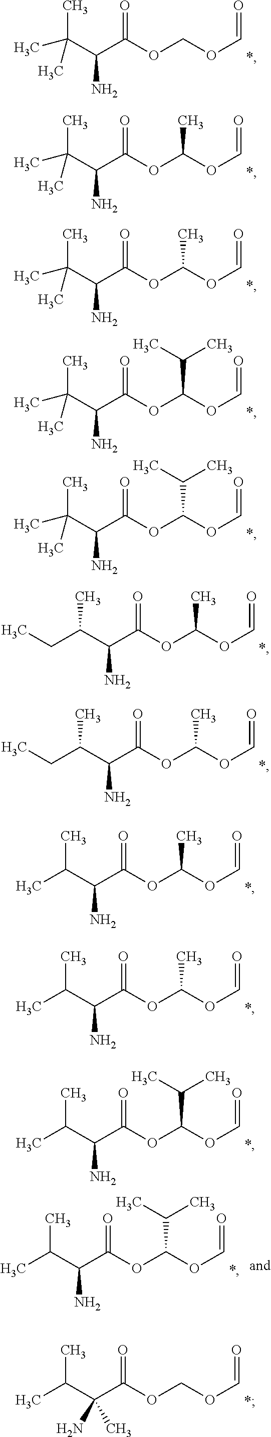

wherein “*” indicates the point of attachment to the nitrogen atom R^{A} is attached to;
   or an N-oxide, a hydrate, a solvate, or a salt thereof, or a mixture of same.

In another preferred embodiment, the invention relates to compounds of the formula (Ia):

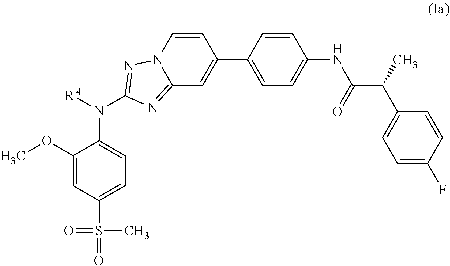

in which:

R^{A} represents a group selected from:

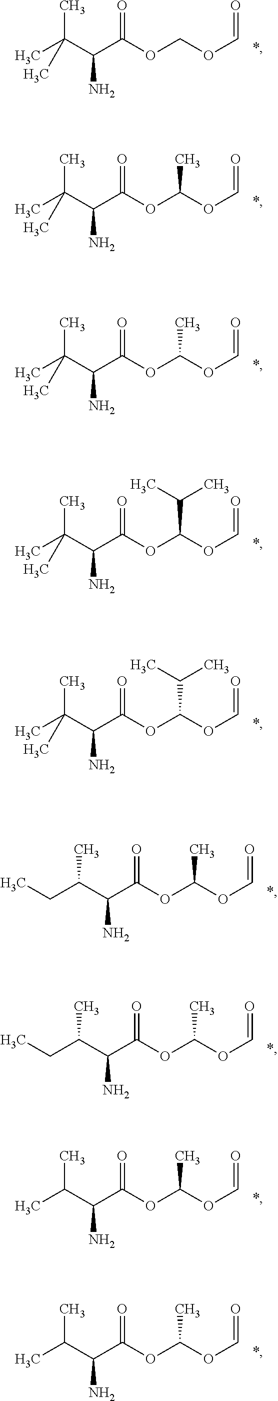

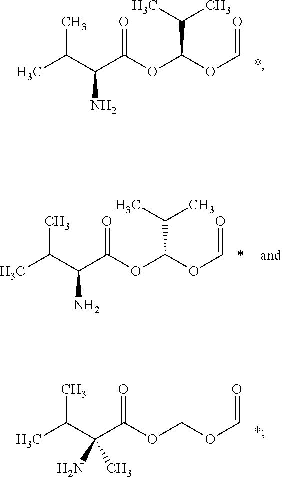

wherein “*” indicates the point of attachment to the nitrogen atom R^{A} is attached to;
or an N-oxide, a hydrate, a solvate, or a salt thereof, or a mixture of same.

In another preferred embodiment, the invention relates to compounds of the formula (Ib):

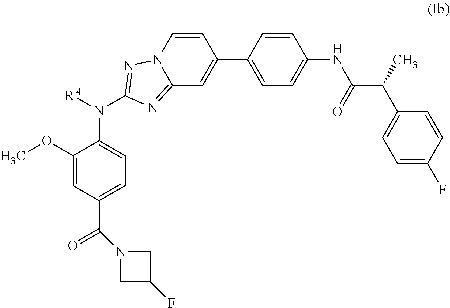

in which:

R^{A} represents a group selected from:

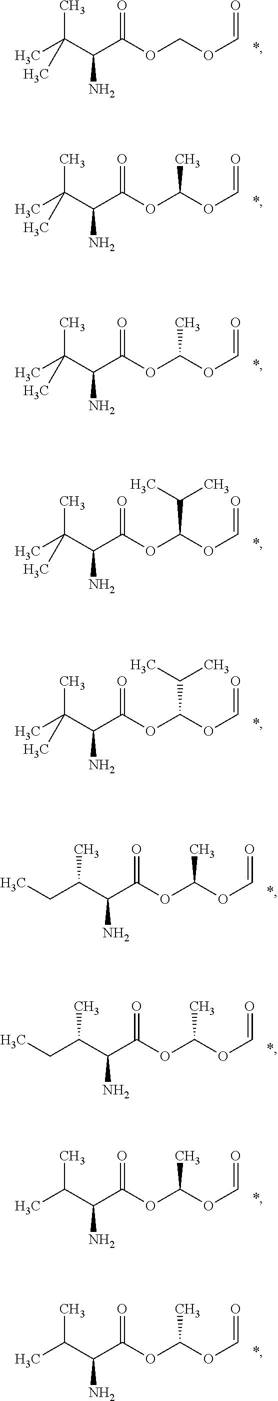

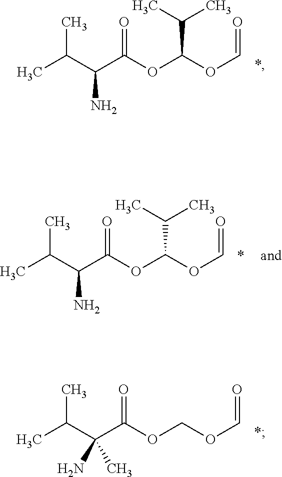

wherein “*” indicates the point of attachment to the nitrogen atom R^{A} is attached to;
or an N-oxide, a hydrate, a solvate, or a salt thereof, or a mixture of same.

In another preferred embodiment, the invention relates to compounds of the formula (Ic):

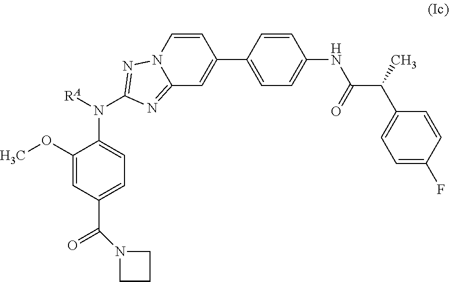

in which:

R^{A} represents a group selected from:

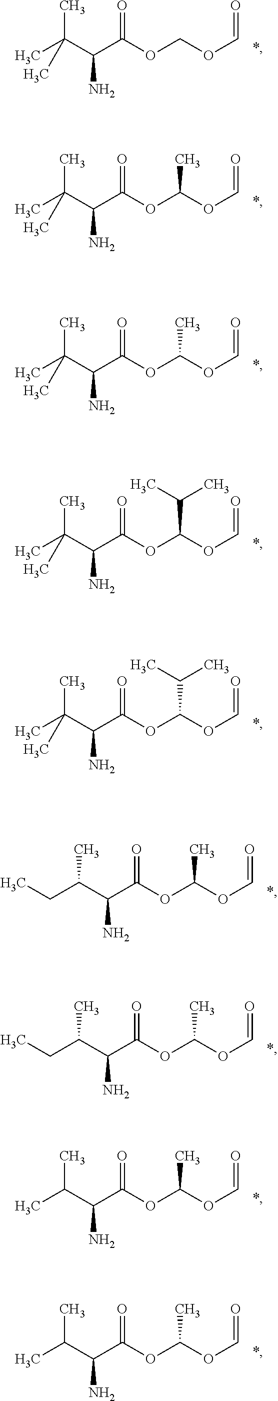

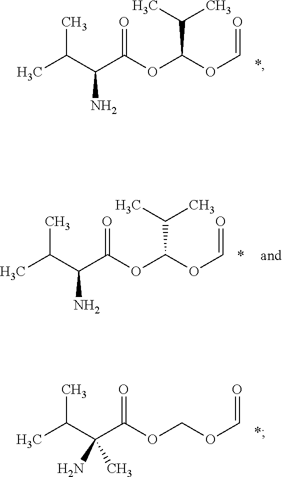

wherein “*” indicates the point of attachment to the nitrogen atom R^{A} is attached to;
or an N-oxide, a hydrate, a solvate, or a salt thereof, or a mixture of same.

In another preferred embodiment, the invention relates to compounds of the formula (Id):

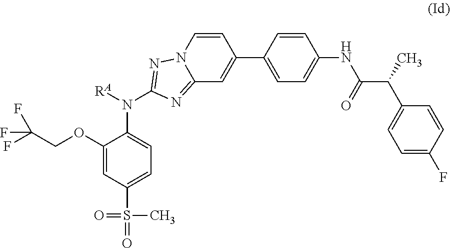

in which:

R^{A} represents a group selected from:

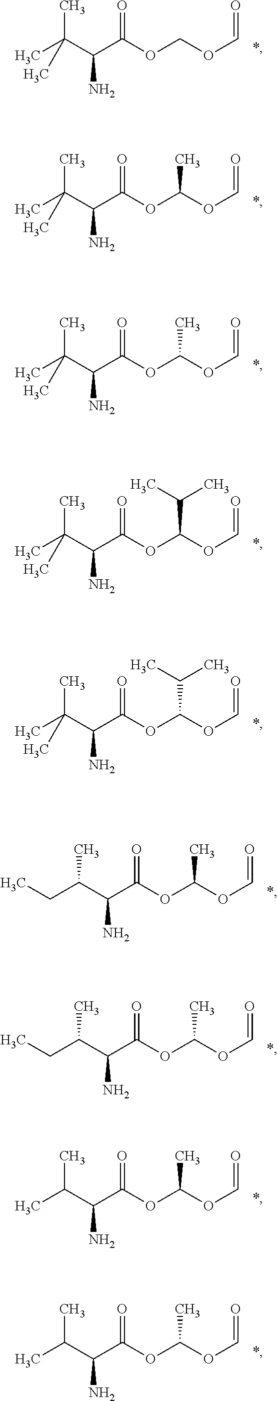

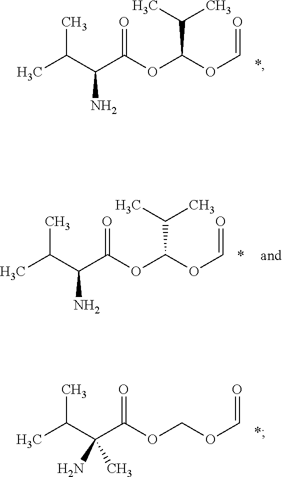

wherein “*” indicates the point of attachment to the nitrogen atom R^{A} is attached to;

or an N-oxide, a hydrate, a solvate, or a salt thereof, or a mixture of same.

In another preferred embodiment, the invention relates to compounds of the formula (Ie):

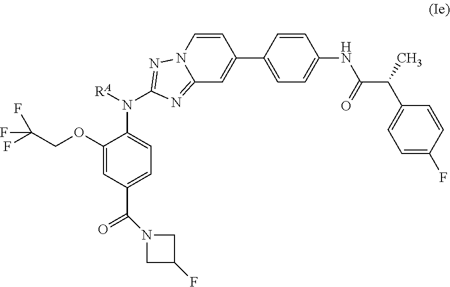

in which:

R^{A} represents a group selected from:

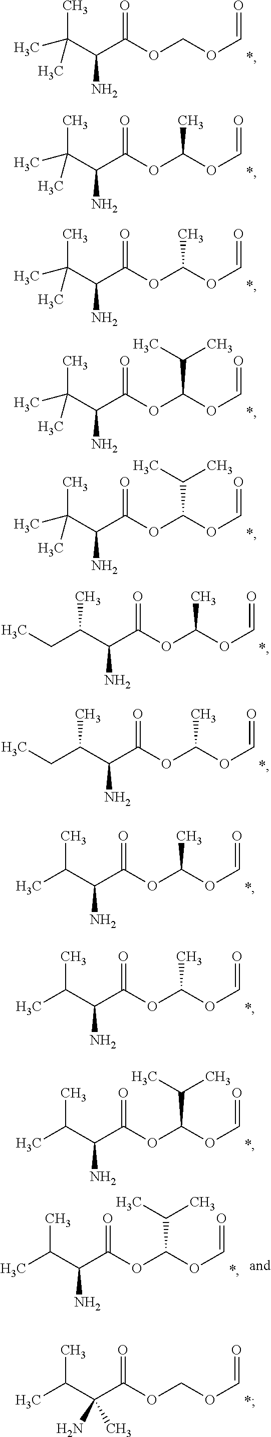

wherein “*” indicates the point of attachment to the nitrogen atom R^{A} is attached to;

or an N-oxide, a hydrate, a solvate, or a salt thereof, or a mixture of same.

In another preferred embodiment, the invention relates to compounds of the formula (If):

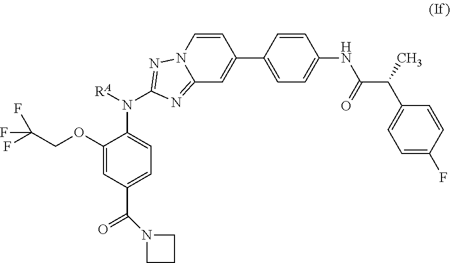

in which:

R^{A} represents a group selected from:

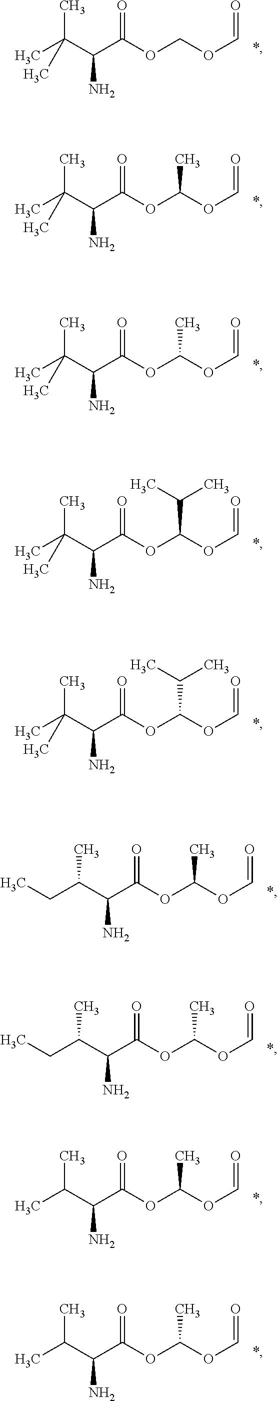

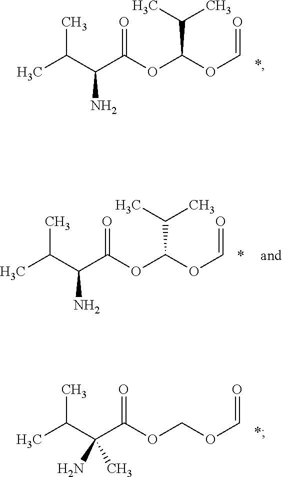

wherein “*” indicates the point of attachment to the nitrogen atom R^{A} is attached to;
or an N-oxide, a hydrate, a solvate, or a salt thereof, or a mixture of same.

It is to be understood that the present invention relates to any sub-combination within any embodiment or aspect of the present invention of compounds of general formula (I), supra.

More particularly still, the present invention covers compounds of general formula (I) which are disclosed in the Examples section of this text, infra.

The invention also includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I respectively. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

Further, the compounds of the present invention can exist as N-oxides, which are defined in that at least one nitrogen of the compounds of the present invention is oxidised. The present invention includes all such possible N-oxides.

The present invention also relates to useful forms of the compounds as disclosed herein, such as hydrates, solvates, salts, in particular pharmaceutically acceptable salts, and co-precipitates.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucannine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, the compounds according to the invention may form salts with a quarternary ammonium ion obtainable e.g. by quarternisation of a basic nitrogen containing group with agents like lower alkylhalides such as methyl-, ethyl-, propyl-, and butylchlorides, -bromides and -iodides; dialkylsulfates like dimethyl-, diethyl-, dibutyl- and diamylsulfates, long chain halides such as decyl-, lauryl-, myristyl- and stearylchlorides, -bromides and -iodides, aralkylhalides like benzyl- and phenethylbromides and others. Examples of suitable quarternary ammonium ions are tetramethylammonium, tetraethylammonium, tetra(n-propyl)ammonium, tetra (n-butyl)ammonium, or N-benzyl-N,N,N-trimethylammonium.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of the compounds of the present invention as single salts, or as any mixture of said salts, in any ratio.

Furthermore, the present invention includes all possible crystalline forms, or polymorphs, of the compounds of the present invention, either as single polymorphs, or as a mixture of more than one polymorphs, in any ratio.

In accordance with another aspect, the present invention covers methods of preparing compounds of the present invention, said methods comprising the steps as described in the Experimental Section herein.

This invention also relates to pharmaceutical compositions containing one or more compounds of the present invention. These compositions can be utilised to achieve the desired pharmacological effect by administration to a patient in need thereof. A patient, for the purpose of this invention, is a mammal, including a human, in need of treatment for the particular condition or disease. Therefore, the present invention includes pharmaceutical compositions that are comprised of a pharmaceutically acceptable carrier and a pharmaceutically effective amount of a compound, or salt thereof, of the present invention. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is preferably that amount which produces a result or exerts an influence on the particular condition being treated.

The compounds of this invention may also be administered parenterally, that is, subcutaneously, intravenously, intraocularly, intrasynovially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

The parenteral compositions of this invention will typically contain from about 0.5% to about 25% by weight of the active ingredient in solution. Preservatives and buffers may also be used advantageously. In order to minimise or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions.

Pharmaceutical compositions according to the present invention can be illustrated as follows:

Sterile i.v. Solution: A 5 mg/mL solution of the desired compound of this invention can be made using sterile, injectable water, and the pH is adjusted if necessary. The solution is diluted for administration to 1-2 mg/mL with sterile 5% dextrose and is administered as an i.v. infusion over about 60 minutes.

As mentioned supra, compound A has been found to effectively inhibit Mps-1 and may therefore be used for the treatment or prophylaxis of diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by Mps-1, such as, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

In accordance with another aspect therefore, the present invention covers a compound of general formula (I), or an N-oxide, a hydrate, a solvate, or a salt thereof, particularly a pharmaceutically acceptable salt thereof, or a mixture of same, as described and defined herein, for use in the treatment or prophylaxis of a disease, as mentioned supra.

Another particular aspect of the present invention is therefore the use of a compound of general formula (I), described supra, or an N-oxide, a hydrate, a solvate, or a salt thereof, particularly a pharmaceutically acceptable salt thereof, or a mixture of same, for the prophylaxis or treatment of a disease.

Another particular aspect of the present invention is therefore the use of a compound of general formula (I) described supra for manufacturing a pharmaceutical composition for the treatment or prophylaxis of a disease.

The term “inappropriate” within the context of the present invention, in particular in the context of “inappropriate cellular immune responses, or inappropriate cellular inflammatory responses”, as used herein, is to be understood as preferably meaning a response which is less than, or greater than normal, and which is associated with, responsible for, or results in, the pathology of said diseases.

The present invention relates to a method for using the compounds of the present invention and compositions thereof, to treat mammalian hyper-proliferative disorders. Compounds can be utilized to inhibit, block, reduce, decrease, etc., cell proliferation and/or cell division, and/or produce apoptosis. This method comprises administering to a mammal in need thereof, including a human, an amount of a compound of this invention, or a pharmaceutically acceptable salt, isomer, polymorph, metabolite, hydrate, solvate or ester thereof; etc. which is effective to treat the disorder. Hyper-proliferative disorders include but are not limited, e.g., psoriasis, keloids, and other hyperplasias affecting the skin, benign prostate hyperplasia (BPH), solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell. Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

The present invention also provides methods for the treatment of disorders associated with aberrant mitogen extracellular kinase activity, including, but not limited to stroke, heart failure, hepatomegaly, cardiomegaly, diabetes, Alzheimer's disease, cystic fibrosis, symptoms of xenograft rejections, septic shock or asthma.

Effective amounts of compounds of the present invention can be used to treat such disorders, including those diseases (e.g., cancer) mentioned in the Background section above. Nonetheless, such cancers and other diseases can be treated with compounds of the present invention, regardless of the mechanism of action and/or the relationship between the kinase and the disorder.

The phrase “aberrant kinase activity” or “aberrant serine-threonine kinase activity,” includes any abnormal expression or activity of the gene encoding the kinase or of the polypeptide it encodes. Examples of such aberrant activity, include, but are not limited to, over-expression of the gene or polypeptide; gene amplification; mutations which produce constitutively-active or hyperactive kinase activity; gene mutations, deletions, substitutions, additions, etc.

The present invention also provides for methods of inhibiting a kinase activity, especially of mitogen extracellular kinase, comprising administering an effective amount of a compound of the present invention, including salts, polymorphs, metabolites, hydrates, solvates, prodrugs (e.g.: esters) thereof, and diastereoisomeric forms thereof. Kinase activity can be inhibited in cells (e.g., in vitro), or in the cells of a mammalian subject, especially a human patient in need of treatment.

### General Synthesis of Prodrug Compounds of Formula (I)

The following paragraphs outline a synthetic approach suitable to prepare compounds of formula (I) as shown in the following scheme.

In addition to the routes described below, also other routes may be used to synthesise the target compounds, in accordance with common general knowledge of a person skilled in the art of organic synthesis. The order of transformations exemplified in the following Schemes is therefore not intended to be limiting, and suitable synthesis steps from various schemes can be combined to form additional synthesis sequences. In addition, interconversion of any of the substituents shown can be achieved before and/or after the exemplified transformations. These modifications can be such as reduction or oxidation of functional groups, halogenation, metallation, metal catalysed coupling reactions, substitution or other reactions known to a person skilled in the art. These transformations include those which introduce a functionality allowing for further interconversion of substituents. In particular, the synthetic routes below encompass the introduction and cleavage of protective groups. Appropriate protective groups and their introduction and cleavage are well-known to a person skilled in the art (see for example T. W. Greene and P. G. M. Wuts in Protective Groups in Organic Synthesis, 4^{th} edition, Wiley 2006); more specifically, protective groups include groups such as PG¹ (protecting group for hydroxy as defined supra), and PG² (protecting group for amino as defined supra).

Specific examples are described in the subsequent paragraphs. Further, it is possible that two or more successive steps may be performed without work-up being performed between said steps, e.g. a “one-pot” reaction, as it is well-known to a person skilled in the art.

Scheme 1 outlines the synthesis of compounds of general formula (I) from intermediates of the formula (V), in which R¹ and R² are as defined for compounds of general formula (I). The preparation of intermediates (V) can be performed as described in the Experimental Section. Intermediates (V) are deprotonated by a suitable base, such as sodium hydride, in a suitable solvent, such as an ether, e.g. tetrahydrofuran, and subsequently reacted with a chloroformiate of formula (VI), in which R⁴ and R⁵ are as defined for compounds of the general formula (I), and LG stands for a leaving group, as defined supra, preferably chloro, to give carbamates (VII). Chloroformiates of formula (VI) are well known to the person skilled in the art, and are commercially available in several cases. Said carbamates (VII) are reacted with a carboxylate salt of the formula (VIII), in which PG² represents a protecting group for amino groups, as defined supra, such as tert-butoxycarbonyl (Boc), benzyloxycarbonyl (Z) or p-methoxybenzyl (PMB), and in which M⁺ stands for a monovalent cation such as an alkali cation or an ammonium salt, preferably cesium, in a suitable solvent, such as N,N-dimethylformamide, to give intermediates of the formula (IX). This substitution can also be performed in the presence of a catalytic amount of an iodide salt like sodium iodide or potassium iodide whereby the leaving group LG is in situ transformed to iodide. Alternatively, the leaving group LG can be transformed to iodide prior to the substitution reaction. Intermediates (IX) are then subjected to a Suzuki coupling involving boronic acid derivatives (X), in which R^{E} stand for hydrogen or independently from each other stand for C₁-C₆-alkyl-, or together form a —C₂-C₆-alkylene-group e.g. —C(CH₃)₂—C(CH₃)₂—. Suzuki couplings are well known to the person skilled in the art; preferably, the coupling is performed using dicyclohexyl(2′,6′-dimethoxybiphenyl-2-yl)phosphine and palladium acetate, or Pd₂dba₃, as ligand/catalyst, potassium phosphate monohydrate or potassium phosphate as a base, and toluene or N-methylpyrrolidine or mixtures of toluene and N-methylpyrrolidine as a solvent. The coupling products (XI) are subsequently deprotected (if needed), e g. by treatment with hydrochloric acid to remove a Boc group, to give compounds of general formula (I).

Compounds of general formula (I) are typically isolated as salts, preferably as HCl salts or as TEA-salts.

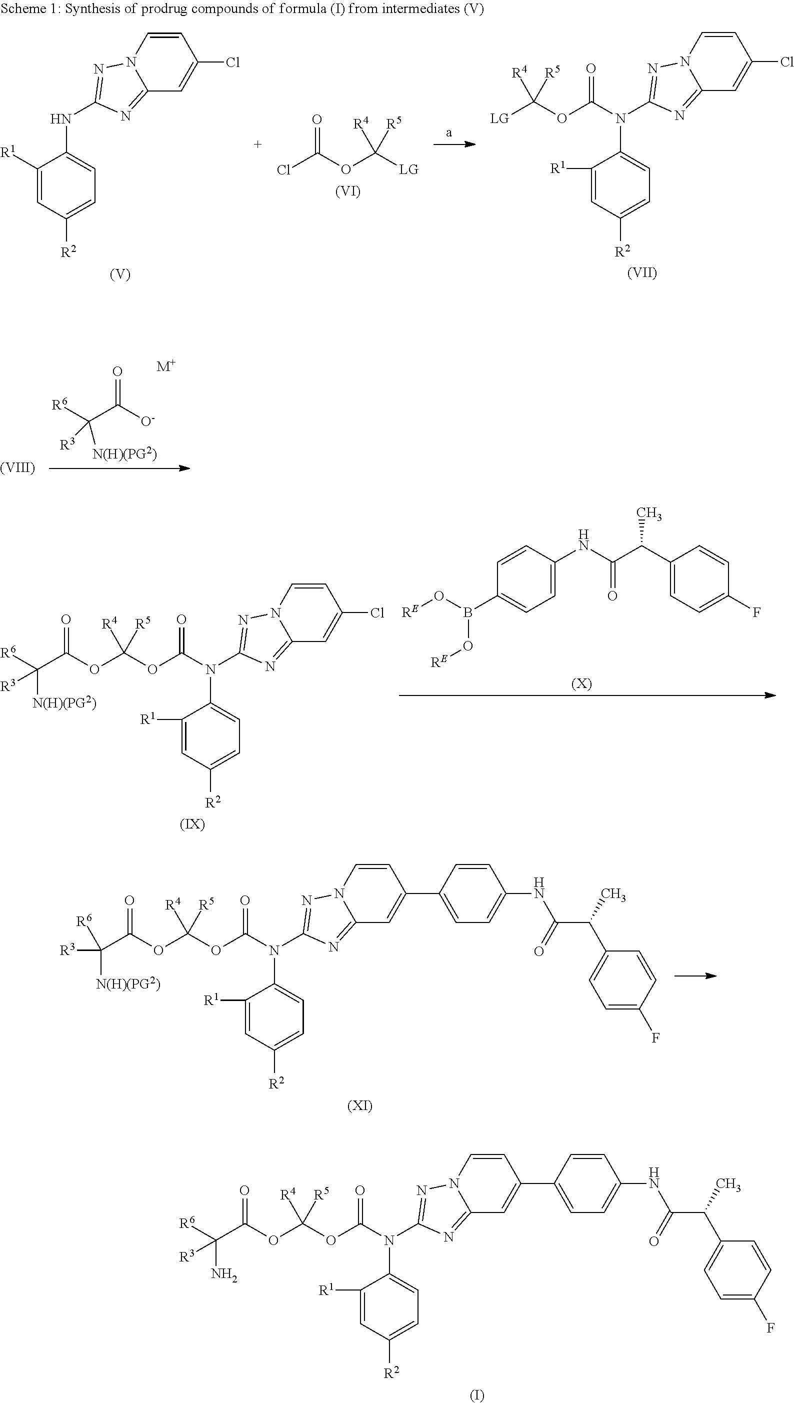

Experimental Section

The following Table lists the abbreviations used in this paragraph, and in the Examples section. NMR peak forms are stated as they appear in the spectra, possible higher order effects have not been considered.

| Abbreviation | Meaning |
|---|---|
| Ac | acetyl |
| BINAP | 2,2′-bis(diphenylphosphino)-1,1′-binaphthyl |
| Boc | tert-butyloxycarbonyl |
| br | broad |
| Brett-Phos | 2-(dicyclohexylphosphino)-3,6-dimethoxy-2′-4′-6′-tri-i-propyl- |
| | 1,1′-biphenyl |
| c- | cyclo- |
| 1-Chloroethyl | 1-chloroethyl carbonochloridate |
| chloroformate | |
| Chloromethyl | chloromethyl carbonochloridate |
| chloroformate | |
| d | doublet |
| dd | doublet of doublets |
| DCM | dichloromethane |
| DME | 1,2-dimethoxyethane |
| DIPE | diisopropylether |
| DIPEA | N,N-diisopropylethylamine |
| DMF | N,N-dimethylformamide |
| DMSO | dimethyl sulfoxide |
| Dppf | 1,1′-bis(diphenylphosphino)ferrocene |
| Eq | equivalent |
| ESI | electrospray ionisation |
| HATU | N-[(dimethylamino)(3H-[1,2,3]triazolo[4,5-b]pyridin-3-yloxy)- |
| | methylene]-N-methylmethanaminium hexafluorophosphate |
| Hunig Base | N,N-diisopropylethylamine |
| m | multiplet |
| m.p. | melting point in ° C. |
| MS | mass spectrometry |
| MW | molecular weight |
| NaOtBu | sodium tert-butoxide; sodium 2-methylpropan-2-olate |
| NMP | N-methylpyrrolidinone |
| NMR | nuclear magnetic resonance spectroscopy: chemical shifts (δ) |
| | are given in ppm. |
| PdCl₂(PPh₃)₂ | dichlorobis(triphenylphosphine)palladium(II) |
| Pd(dba)₂ | bis-(dibenzylideneacetone)palladium(0) complex |
| Pd₂(dba)₃ | tris-(dibenzylideneacetone)dipalladium(0) chloroform |
| | complex |
| Pd(dppf)Cl₂ | dichloro[1,1′-bis(diphenylphosphino)ferrocene]palladium(II) |
| Pd(dppf)Cl₂•CH₂Cl₂ | dichloro[1,1′-bis(diphenylphosphino)ferrocene]palladium(II) |
| | dichloromethane adduct |
| Pd-Brett-Phos- | chloro[2-(dicyclohexylphosphino)-3,6-dimethoxy-2′-4′-6′-tri- |
| pre-cat | iso-propyl-1,1′-biphenyl][2-(2- |
| | aminoethyl)phenyl]palladium(II) |
| Pd-tBu-X-Phos- | chloro(2-di-tert-butylphosphino-2′,4′,6′-tri-isopropyl-1,1′- |
| pre-cat | biphenyl)[2-(2-aminoethyl)phenyl] palladium(II), |
| Pd-X-Phos-pre- | chloro(2-dicyclohexylphosphino-2′,4′,6′-tri-isopropyl-1,1′- |
| cat | biphenyl)[2-(2-aminoethyl)phenyl] palladium(II) methyl-tert- |
| | butylether adduct |
| PPh₃ | triphenylphosphine |
| P(oTol)₃ | tri-o-tolylphosphine |
| q | quartet |
| quin | quintett |
| Rac | racemic |
| Rt | room temperature |
| r.t. | room temperature |
| RT | retention time in minutes |
| s | singlet |
| S-Phos | dicyclohexyl(2′,6′-dimethoxybiphenyl-2-yl)phosphine |
| t | triplet |
| TBAF | tetrabutylammoniumfluoride |
| tBu-X-Phos | 2-di-tert-butylphosphino-2′,4′,6′-tri-isopropyl-1,1′-biphenyl |
| TBDPS | tert-butyldiphenylsilyl |
| TBTU | N-[(1H-benzotriazol-1-yloxy)(dimethylamino)methylene]-N- |
| | methylmethanaminium tetrafluoroborate |
| TEA | triethylamine |
| TFA | trifluoroacetic acid |
| THF | tetrahydrofuran |
| TMS | trimethylsilyl |
| Ts | para toluenesulfonyl; (tosyl) |
| UPLC | ultra performance liquid chromatography |
| X-Phos | 2-dicyclohexylphosphino-2′,4′,6′-triisopropylbiphenyl |

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallisation. In some cases, impurities may be stirred out using a suitable solvent. In some cases, the compounds may be purified by chromatography, particularly flash chromatography, using for example pre-packed silica gel cartridges, e.g. from Separtis such as Isolute® Flash silica gel (silica gel chromatography) or Isolute® Flash NH2 silica gel (aminophase-silica-gel chromatography) in combination with a suitable chromatographic system such as a Flashmaster II (Separtis) or an Isolera system (Biotage) and eluents such as, for example, gradients of hexane/ethyl acetate or DCM/methanol. In some cases, the compounds may be purified by preparative HPLC using, for example, a Waters autopurifier equipped with a diode array detector and/or on-line electrospray ionisation mass spectrometer in combination with a suitable pre-packed reverse phase column and eluants such as, for example, gradients of water and acetonitrile which may contain additives such as trifluoroacetic acid, formic acid or aqueous ammonia.

Optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Diacel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae such as “hydrochloride”, “trifluoroacetate”, “sodium salt”, or “x HCl”, “x CF₃COOH”, “x Na⁺”, for example, are to be understood as not a stoichiometric specification, but solely as a salt form.

This applies analogously to cases in which synthesis intermediates or example compounds or salts thereof have been obtained, by the preparation and/or purification processes described, as solvates, such as hydrates with (if defined) unknown stoichiometric composition.

The IUPAC names of the examples and intermediates were generated using the program ‘ACD/Nanne batch version 12.01’ from ACD LABS, and were adapted if needed.

Analytical UPLC-MS was performed as follows:

LC-MS methods:

Method 1:

Instrument: Waters Acquity UPLCMS ZQ4000; Column: Acquity UPLC BEH C18 1.7 μm, 50×2.1 mm; eluent A: water+0.05 vol % formic acid, Eluent B: acetonitrile+0.05 vol % formic acid gradient: 0-1.6 min 1-99% B, 1.6-2.0 min 99% B; flow 0.8 mL/min; temperature: 60° C.; injection: 2 μL; DAD scan: 210-400 nm; ELSD.

Method 2:

Instrument: Waters Acquity UPLC-MS SQD 3001; Column: Acquity UPLC BEH C18 1.7 μm, 50×2.1 mm; eluent A: water+0.1 vol % formic acid (95%), eluent B: acetonitrile, gradient: 0-1.6 min 1-99% B, 1.6-2.0 min 99% B; flow 0.8 mL/min; temperature: 60° C.; injection: 2 μL; DAD scan: 210-400 nm; ELSD.

Method 3: Instrument: Waters Acquity UPLCMS SQD; Column: Acquity UPLC BEH C18 1.7 μm, 50×2.1 mm; eluent A: water+0.05 vol % formic acid (95%), eluent B: acetonitrile+0.05 vol % formic acid (95%), gradient: 0-1.6 min 1-99% B, 1.6-2.0 min 99% B; flow 0.8 mL/min; temperature: 60° C.; injection: 2 μL; DAD scan: 210-400 nm; ELSD.

Method 4:

Instrument: Waters Acquity UPLC-MS SQD; Column: Acquity UPLC BEH C18 1.7 50×2.1 mm; eluent A: water+0.1 vol % formic acid (99%), eluent B: acetonitrile; gradient: 0-1.6 min 1-99% B, 1.6-2.0 min 99% B; flow 0.8 mL/min; temperature: 60° C.; injection: 2 μL; DAD scan: 210-400 nm; ELSD.

Method 5: Instrument: Waters Acquity UPLCMS SQD 3001; Column: Acquity UPLC BEH C18 1.7 μm, 50×2.1 mm; eluent A: water+0.2 vol. % ammonia (32%), eluent B: acetonitrile, gradient: 0-1.6 min 1-99% B, 1.6-2.0 min 99% B; flow 0.8 mL/min; temperature: 60° C.; injection: 2 μL; DAD scan: 210-400 nm; ELSD.

Method 6

Instrument: Waters Acquity UPLC-MS SQD; Column: Acquity UPLC BEH C18 1.7 50×2.1 mm; eluent A: water+0.2% vol. ammonia (32%), eluent B: acetonitrile; gradient: 0-1.6 min 1-99% B, 1.6-2.0 min 99% B; flow 0.8 mL/min; temperature: 60° C.; injection: 2 μl; DAD scan: 210-400 nm; ELSD.

Method 7

Instrument: Waters Acquity UPLC-MS ZQ; column: Acquity UPLC BEH C18 1.7 50×2.1 mm; eluent A: water+0.1% vol. formic acid (99%), eluent B: acetonitrile; gradient: 0-1.6 min 1-99% B, 1.6-2.0 min 99% B; flow 0.8 mL/min; temperature: 60° C.; injection: 2 μl; DAD scan: 210-400 nm; ELSD.

Method 8:

Instrument: Waters Acquity UPLCMS SQD; Column: Acquity UPLC BEH C18 1.7 μm, 50×2.1 mm; Eluent A: water+0.2% vol. ammonia (32%), eluent B: acetonitrile; gradient: 0-1.6 min 1-99% B, 1.6-2.0 min 99% B; flow 0.8 mL/min; temperature: 60° C.; injection: 2 μl; DAD scan: 210-400 nm; ELSD.

### INTERMEDIATE EXAMPLE 01.01

### ethyl [(4-chloropyridin-2-yl)carbamothioyl]carbamate

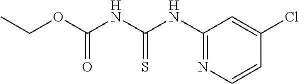 [SMILES: CCOC(=O)NC(=S)Nc1cc(Cl)ccn1]

Ethoxycarbonyl isothiocyanate (11.1 g) was added to a stirred solution of 2-amino-4-chloropyridine (10.1 g) in dioxane (100 mL). The mixture was stirred for 2 h at r.t. A white solid precipitated. Hexane (25 mL) was added and the white solid was collected by filtration to give 8.0 g of the title compound. The solution was concentrated in vacuum and the residue was recrystallized from ethyl acetate to give further 8.5 g of the title compound.

### INTERMEDIATE EXAMPLE 01.02

### 7-chloro[1,2,4]triazolo[1,5-a]pyridin-2-amine

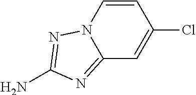 [SMILES: Nc1nc2cc(Cl)ccn2n1]

Hydroxylammonium chloride (13.9 g) was suspended in methanol (70 mL), and ethanol (65 mL) and Hünig Base (21.1 mL) were added at r.t. The mixture was heated to 60° C., ethyl [(4-chloropyridin-2-yl)carbamothioyl]carbamate (9.0 g) was added portionwise, and the mixture was stirred at 60° C. for 2 h. The solvent was removed in vacuum and water (150 mL) was added. A solid was collected by filtration and was washed with ethanol and dried in vacuum. Silicagel chromatography gave 4.2 g of the title compound.

¹H-NMR (300 MHz, DMSO-d₆), δ [ppm]=6.14 (2H), 6.92 (1H), 7.50 (1H), 8.55 (1H).

### INTERMEDIATE EXAMPLE 01.03

### {4-[(7-chloro[1,2,4]triazolo[1,5-a]pyridin-2-yl)amino]-3-methoxyphenyl}(3-fluoroazetidin-1-yl)methanone

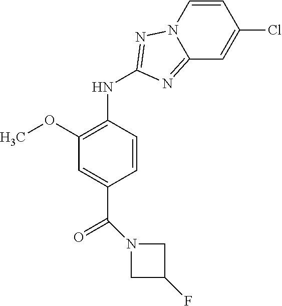 [SMILES: COc1cc(C(=O)N2CC(F)C2)ccc1Nc1nc2cc(Cl)ccn2n1]

To a stirred suspension of 7-chloro[1,2,4]triazolo[1,5-a]pyridin-2-amine (190 mg) in toluene (7 mL) and NMP (0.7 mL) was added (4-Bromo-3-methoxyphenyl)(3-fluoroazetidin-1-yl)methanone (373 mg), chloro(2-dicyclohexylphosphino-2′,4′,6′-tri-iso-propyl-1,1′-biphenyl)[2-(2-aminoethyl) phenyl]palladium(II) methyl-tert-butylether adduct (28 mg), X-Phos (16 mg) and powdered potassium phosphate monohydrate (0.60 g) and the flask was degassed twice and backfilled with argon. The mixture was heated to reflux for 16 h. A half-saturated solution of potassium carbonate was added and the mixture was extracted with a mixture of dichloromethane and methanol. The organic phase was dried (sodium sulfate) and the solvent was removed in vacuum. The mixture was filtered and concentrated in vacuum. Silicagel chromatography gave 120 mg of the title compound.

¹H-NMR (300 MHz, DMSO-d₆), δ [ppm]=3.91 (3H), 3.94-4.80 (4H), 5.26-5.59 (1H), 7.15 (1H), 7.23-7.33 (2H), 7.82 (1H), 8.21-8.36 (1H), 8.46 (1H), 8.85 (1H).

### INTERMEDIATE EXAMPLE 01.04

### 7-chloro-N-[2-methoxy-4-(methylsulfonyl)phenyl][1, 2,4]triazolo[1,5-a]pyridin-2-amine

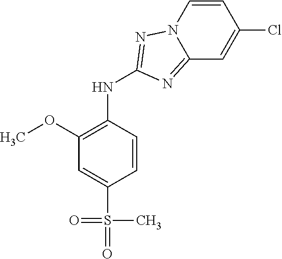 [SMILES: COc1cc(S(C)(=O)=O)ccc1Nc1nc2cc(Cl)ccn2n1]

Starting from 7-chloro[1,2,4]triazolo[1,5-a]pyridin-2-amine (300 mg) and 1-bromo-2-methoxy-4-(methylsulfonyl)benzene (543 mg), Intermediate Example 01.04. was prepared analogously to the procedure for the preparation of Intermediate Example 01.03. Yield: 236 mg of the title compound.

¹H-NMR (300 MHz, DMSO-d₆), δ [ppm]=3.18 (3H), 3.97 (3H), 7.17 (1H), 7.44 (1H), 7.53 (1H), 7.86 (1H), 8.43 (1H), 8.75 (1H), 8.87 (1H).

### INTERMEDIATE EXAMPLE 01.05

### 7-chloro-N-[4-(methylsulfonyl)-2-(2,2,2-trifluoroethoxy)phenyl][1,2,4]triazolo[1,5-a]pyridin-2-amine

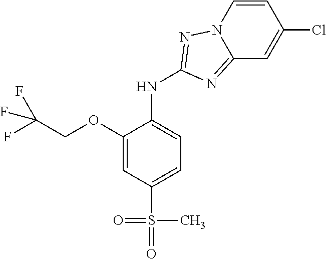 [SMILES: CS(=O)(=O)c1ccc(Nc2nc3cc(Cl)ccn3n2)c(OCC(F)(F)F)c1]

Starting from 7-chloro[1,2,4]triazolo[1,5-a]pyridin-2-amine (100 mg) and 1-bromo-4-(methylsulfonyl)-2-(2,2,2-trifluoroethoxy)benzene (227 mg), Intermediate Example 01.05. was prepared analogously to the procedure for the preparation of Intermediate Example 01.03. Yield: 50 mg of the title compound.

¹H-NMR (400 MHz, DMSO-d₆), δ [ppm]=3.19 (3H), 5.00 (2H), 7.18 (1H), 7.58-7.71 (2H), 7.86 (1H), 8.44 (1H), 8.70 (1H), 8.81-8.92 (1H).

### INTERMEDIATE EXAMPLE 01.06

### {4-[(7-chloro[1,2,4]triazolo[1,5-a]pyridin-2-yl)amino]-3-(2,2,2-trifluoroethoxy)phenyl}(3-fluoroazetidin-1-yl)methanone

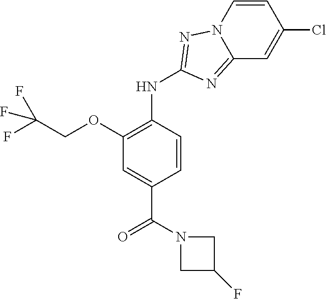 [SMILES: O=C(c1ccc(Nc2nc3cc(Cl)ccn3n2)c(OCC(F)(F)F)c1)N1CC(F)C1]

Starting from 7-chloro[1,2,4]triazolo[1,5-a]pyridin-2-amine (250 mg) and [4-bromo-3-(2,2,2-trifluoroethoxy)phenyl](3-fluoroazetidin-1-yl)methanone (607 mg), Intermediate Example 01.06. was prepared analogously to the procedure for the preparation of Intermediate Example 01.03. Yield: 198 mg of the title compound.

¹H-NMR (400 MHz, DMSO-d₆), δ [ppm]=3.93-4.72 (4H), 4.93 (2H), 5.32-5.55 (1H), 7.16 (1H), 7.36-7.43 (2H), 7.83 (1H), 8.27-8.33 (1H), 8.41 (1H), 8.81-8.90 (1H)

### INTERMEDIATE EXAMPLE 01.07

### azetidin-1-yl{4-[(7-chloro[1,2,4]triazolo[1,5-a]pyridin-2-yl)amino]-3-methoxyphenyl}methanone

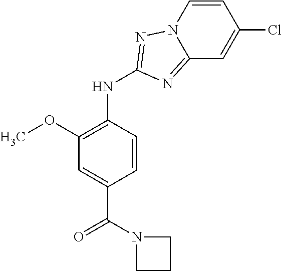 [SMILES: COc1cc(C(=O)N2CCC2)ccc1Nc1nc2cc(Cl)ccn2n1]

Starting from 7-chloro[1,2,4]triazolo[1,5-a]pyridin-2-amine (190 mg) and azetidin-1-yl(4-bromo-3-methoxyphenyl)methanone (350 mg), Intermediate Example 01.07. was prepared analogously to the procedure for the preparation of Intermediate Example 01.03. Yield: 130 mg of the title compound.

¹H-NMR (400 MHz, DMSO-d₆), δ [ppm]=2.27 (2H), 3.88-3.94 (3H), 3.97-4.47 (4H), 7.15 (1H), 7.23-7.31 (2H), 7.83 (1H), 8.28 (1H), 8.42 (1H), 8.79-8.93 (1H).

### INTERMEDIATE EXAMPLE 02.01

### Rac-methyl 2-(4-fluorophenyl)propanoate

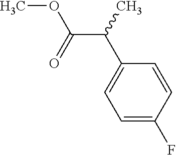

To a stirred solution of diisopropylamine (13.0 g) in tetrahydrofurane (160 mL) was added a solution of n-butyllithium in hexane (51.4 mL; c=2.5 M) at −78° C. The solution was stirred at 0° C. for 15 minutes. The solution was cooled to −78° C. and a solution of methyl (4-fluorophenyl)acetate (18.0 g), dissolved in tetrahydrofurane (40 mL) was added. The solution was stirred at −78° C. for 30 minutes. Methyl iodide (10.0 mL) was added at −78° C., and the solution was allowed to warm up to 0° C. within 1 h. Water was added and the reaction mixture was extracted with ethyl acetate. The organic phase was dried (sodium sulfate) and the solvent was removed in vacuum. Silicagel chromatography gave 18.9 g of the title compound.

¹H-NMR (400MHz, DMSO-d6): δ [ppm]=1.34 (d, 3H), 3.55 (s, 3H), 3.79 (q, 1H), 7.08-7.15 (m, 2H), 7.25-7.32 (m, 2H).

### INTERMEDIATE EXAMPLE 02.02

### Rac-2-(4-fluorophenyl)propanoic acid

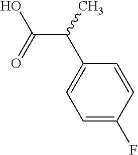

To a stirred solution of Intermediate Example 02.01. (18.9 g) in ethanol (200 mL) was added a solution of potassium hydroxide (35 g), dissolved in water (200 mL). The mixture was stirred at 0° C. for 4 h. Hydrochloric acid (c=4.0 M) was added until pH 5 was reached and the reaction mixture was extracted with ethyl acetate. The organic phase was separated and the solvent was removed in vacuum to give 15.64 g of the title product. The crude product was used without further purification.

¹H-NMR (300MHz, DMSO-d₆): δ [ppm]=1.31 (d, 3H), 3.66 (q, 1H), 7.05-7.15 (m, 2H), 7.24-7.33 (m, 2H), 12.30 (s, 1H).

### INTERMEDIATE EXAMPLE 02.03

### (2R)-2-(4-fluorophenyl)propanoic acid

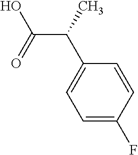

To a stirred solution of Intermediate Example 02.02. (23.6 g) in refluxing ethyl acetate (250 mL) was added a solution of (1S)-1-phenylethanamine (17.35 g) in ethyl acetate. The mixture was allowed to cool down to room temperature within 1 h. A white solid was collected by filtration, was washed with ethyl acetate and dried in vacuum to give 27.5 g of a solid. The solid was recrystallized from 400 mL refluxing ethyl acetate. The mixture was allowed to cool down to room temperature. A white solid was collected by filtration, was washed with ethyl acetate and dried in vacuum to give 18.3 g of a solid. The solid was twice recrystallized from refluxing ethyl acetate (350 mL; 300 mL). A white solid was collected by filtration, was washed with ethyl acetate and dried in vacuum to give 10.51 g of a solid. The solid was dissolved in water, hydrochloric acid (c=2.0 M) was added until pH 5 was reached and the reaction mixture was extracted with dichloromethane. The organic phase was dried (sodium sulfate) and the solvent was removed in vacuum to give 5.6 g of the title product. The crude product was used without further purification.

¹H-NMR (300MHz, DMSO-d₆): δ [ppm]=1.31 (d, 3H), 3.66 (q, 1H), 7.05-7.16 (m, 2H), 7.24-7.33 (m, 2H), 12.28 (br. s., 1H). [α]_{D}²⁰: −79.3° (in DMSO)

Determination of enantiomeric purity by analytical chiral HPLC:

Column: Chiralcel OJ-H 150×4.6; Flow: 1.00 mL/min; Solvent: A: Hexane, B: 2-propanol with 0.1% formic acid; Solvent mixture: 80% A+20% B. Run Time: 30 min. Retention Time: 3.41 min; UV 254 nm; Enantiomeric Ratio: 99.8%: 0.2%.

### INTERMEDIATE EXAMPLE 02.04

### (2R)-2-(4-fluorophenyl)-N-[4-(4,4,5,5-tetramethyl-1,3,2-dioxaborolan-2-yl)phenyl]propanamide

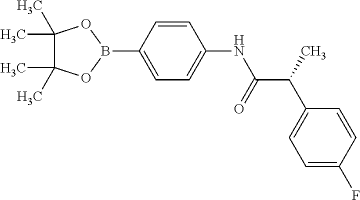 [SMILES: C[C@@H](C(=O)Nc1ccc(B2OC(C)(C)C(C)(C)O2)cc1)c1ccc(F)cc1]

To a stirred solution of 4-(4,4,5,5-tetramethyl-1,3,2-dioxaborolan-2-yl)aniline (1.0 g) in DMF (45 mL) and dichloromethane (90 mL) was added sodium bicarbonate (766 mg), (2R)-2-(4-fluorophenyl)propanoic acid (844 mg) and HATU (2.6 g). The mixture was stirred at room temperature for 4 h. Water was added, and the mixture was stirred for 30 minutes. A half-saturated solution of sodium bicarbonate was added and the mixture was extracted with ethyl acetate. The organic phase was washed with saturated sodium chloride solution, dried (sodium sulfate) and the solvent was removed in vacuum. Silica-gel chromatography gave 1.53 g of the title compound.

¹H-NMR (400 MHz, DMSO-d₆), δ [ppm]=1.23 (12H), 1.37 (3H), 3.74-3.87 (1H), 7.06-7.16 (2H), 7.31-7.42 (2H), 7.51-7.61 (4H), 10.12 (1H).

### INTERMEDIATE EXAMPLE 02.05

### (4-{[(2R)-2-(4-fluorophenyl)propanoyl]amino}phenyl)boronic acid

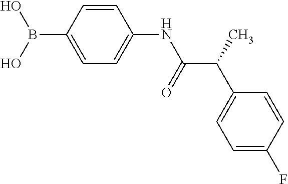 [SMILES: C[C@@H](C(=O)Nc1ccc(B(O)O)cc1)c1ccc(F)cc1]

To a stirred solution of (4-aminophenyl)boronic acid hydrochloride (2.00 g) in DMF (42 mL) was added sodium bicarbonate (2.9 g), (2R)-2-(4-fluorophenyl)propanoic acid (2.04 g) and HATU (6.58 g). The mixture was stirred at room temperature for 72 h. Water (140 mL) was added, and the mixture was stirred for 2 h. The white precipitate was collected by filtration and was washed with water and was dried in vacuum to give 2.86 g of the title compound.

¹H-NMR (300 MHz, DMSO-d₆), δ [ppm]=1.39 (3H), 3.84 (1H), 7.08-7.21 (2H), 7.35-7.44 (2H), 7.52 (2H), 7.69 (2H), 7.88 (2H), 10.07 (1H).

### INTERMEDIATE EXAMPLE 02.06

### (2R)—N-[4-(2-amino[1,2,4]triazolo[1,5-a]pyridin-7-yl)phenyl]-2-(4-fluorophenyl)propanamide

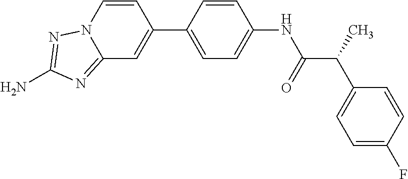 [SMILES: C[C@@H](C(=O)Nc1ccc(-c2ccn3nc(N)nc3c2)cc1)c1ccc(F)cc1]

To a stirred solution of 7-bromo[1,2,4]triazolo[1,5-a]pyridin-2-amine (100 mg; CAS-RN [882521-63-3]; commercially available from Allichem LLC, USA; Baltimore, Md.; preparation described WO2010/020363A1) in 1-propanol (3 mL) was added potassium carbonate solution (0.7 mL, c=2 M), (4-{[(2R)-2-(4-fluorophenyl)propanoyl]amino}phenyl)boronic acid (202 mg), triphenylphosphine (12 mg) and PdCl₂(PPh₃)₂ (33 mg). The mixture was heated to reflux for 16 h. Further triphenylphosphine (12 mg) and PdCl₂(PPh₃)₂ (33 mg) were added and the mixture was heated to reflux for further 4 h. The reaction mixture was filtered through an aminophase-silica-gel column and the solvent was removed in vacuum. Silicagel chromatography gave 150 mg of the title compound.

¹H-NMR (400 MHz, DMSO-d₆), δ [ppm]=1.42 (3H), 3.86 (1H), 5.97 (2H), 7.08-7.25 (3H), 7.35-7.49 (2H), 7.58 (1H), 7.63-7.83 (4H), 8.53 (1H), 10.21 (1H).

### REFERENCE EXAMPLE 01.01

### (2R)-2-(4-fluorophenyl)-N-[4-(2-{[2-methoxy-4-(methylsulfonyl)phenyl]amino}[1,2,4]triazolo[1,5-a]pyridin-7-yl)phenyl]propanamide

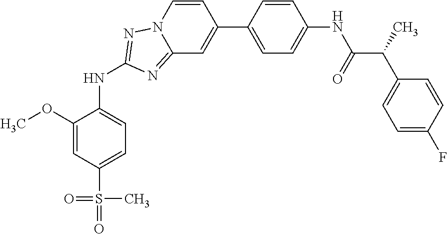 [SMILES: COc1cc(S(C)(=O)=O)ccc1Nc1nc2cc(-c3ccc(NC(=O)[C@H](C)c4ccc(F)cc4)cc3)ccn2n1]

To a stirred suspension of (2R)—N-[4-(2-annino[1,2,4]triazolo[1,5-a]pyridin-7-yl)phenyl]-2-(4-fluorophenyl)propanamide (100 mg) in toluene (4 mL) and NMP (0.2 mL) was added 1-bromo-2-methoxy-4-(methylsulfonyl)benzene (106 mg), chloro(2-dicyclohexylphosphino-2′,4′,6′-tri-iso-propyl-1,1′-biphenyl)[2-(2-aminoethyl)phenyl]palladium(II) methyl-tert-butylether adduct (22 mg), X-Phos (13 mg) and powdered potassium phosphate monohydrate (283 mg) and the flask was degassed twice and backfilled with argon. The mixture was heated to reflux for 16 h. The mixture was filtered and concentrated in vacuum. Silicagel chromatography followed by preparative reverse phase HPLC gave 10 mg of the title compound.

¹H-NMR (400 MHz, DMSO-d₆), δ [ppm]=1.44 (3H), 3.20 (3H), 3.88 (1H), 4.00 (3H), 7.12-7.24 (2H), 7.40-7.50 (4H), 7.56 (1H), 7.75 (2H), 7.86 (2H), 7.92 (1H), 8.52 (1H), 8.63 (1H), 8.86 (1H), 10.28 (1H).

### REFERENCE EXAMPLE 01.02

### (2R)—N-{4-[2-({4-[(3-fluoroazetidin-1-yl)carbonyl]-2-methoxyphenyl}amino)[1,2,4]triazolo[1,5-a]pyridin-7-yl]phenyl}-2-(4-fluorophenyl)propanamide

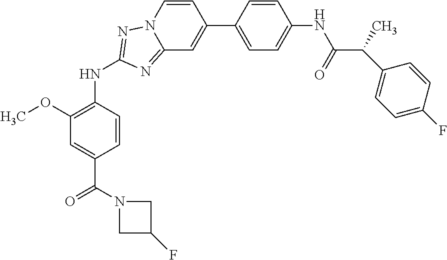 [SMILES: COc1cc(C(=O)N2CC(F)C2)ccc1Nc1nc2cc(-c3ccc(NC(=O)[C@H](C)c4ccc(F)cc4)cc3)ccn2n1]

To a stirred suspension of {4-[(7-chloro[1,2,4]triazolo[1,5-a]pyridin-2-yl)annino]-3-methoxyphenyl}(3-fluoroazetidin-1-yl)methanone (110 mg) in toluene (4.0 mL) and NMP (0.4 mL) was added (4-{[(2R)-2-(4-fluorophenyl)propanoyl]amino}phenyl)boronic acid (126 mg), powdered potassium phosphate monohydrate (248 mg), dicyclohexyl(2′,6′-dimethoxybiphenyl-2-yl)phosphine (24 mg) and Pd(OAc)₂ (6.6 mg) and the flask was degassed twice and backfilled with argon. The mixture was heated to reflux for 2 h. The reaction mixture was filtered and the solvent was removed in vacuum. Aminophase silicagel chromatography gave a solid that was triturated with ether to give 150 mg of the title compound.

¹H-NMR (400 MHz, DMSO-d₆), δ [ppm]=1.44 (3H), 3.82-3.98 (4H), 3.98-4.77 (4H), 5.31-5.59 (1H), 7.18 (2H), 7.24-7.35 (2H), 7.37-7.50 (3H), 7.75 (2H), 7.80-7.95 (3H), 8.29-8.48 (2H), 8.83 (1H), 10.27 (1H).

### REFERENCE EXAMPLE 01.03

### (2R)—N-{4-[2-({4-[(3-fluoroazetidin-1-yl)carbonyl]-2-(2,2,2-trifluoroethoxy)phenyl}amino)[1,2,4]triazolo[1,5-a]pyridin-7-yl]phenyl}-2-(4-fluorophenyl)propanamide

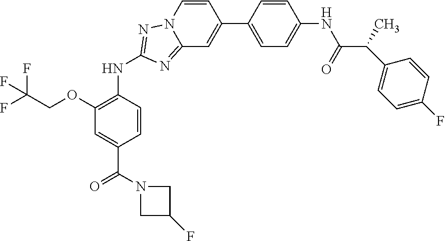 [SMILES: C[C@@H](C(=O)Nc1ccc(-c2ccn3nc(Nc4ccc(C(=O)N5CC(F)C5)cc4OCC(F)(F)F)nc3c2)cc1)c1ccc(F)cc1]

Starting from {4-[(7-chloro[1,2,4]triazolo[1,5-a]pyridin-2-yl)amino]-3-(2,2,2-trifluoroethoxy)phenyl}(3-fluoroazetidin-1-yl)methanone (70 mg) and (4-{[(2R)-2-(4-fluorophenyl)propanoyl]amino}phenyl)boronic acid (61 mg), Reference Example 01.03. was prepared analogously to the procedure for the preparation of Reference Example 01.02. Yield: 73 mg of the title compound.

¹H-NMR (400 MHz, DMSO-d₆), δ [ppm]=1.44 (3H), 3.89 (1H), 3.96-4.76 (4H), 4.96 (2H), 5.34-5.59 (1H), 7.13-7.22 (2H), 7.39-7.48 (5H), 7.75 (2H), 7.81-7.87 (2H), 7.89 (1H), 8.28 (1H), 8.38-8.44 (1H), 8.84 (1H), 10.28 (1H).

### REFERENCE EXAMPLE 01.04

### (2R)-2-(4-fluorophenyl)-N-(4-{2-[(6-methoxy-1,1-dioxido-2,3-dihydro-1-benzothiophen-5-yl)amino][1,2,4]triazolo[1,5-a]pyridin-7-yl}phenyl)propanamide

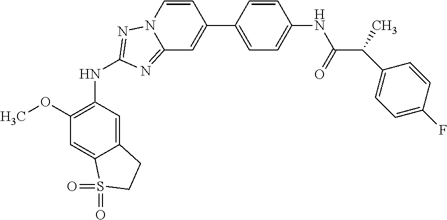 [SMILES: COc1cc2c(cc1Nc1nc3cc(-c4ccc(NC(=O)[C@H](C)c5ccc(F)cc5)cc4)ccn3n1)CCS2(=O)=O]

The compound of Reference Example 01.04. can be prepared in analogy to the methods described herein.

### REFERENCE EXAMPLE 01.05

### (2R)-2-(4-fluorophenyl)-N-[4-(2-{[4-(methylsulfonyl)-2-(2,2,2-trifluoroethoxy)phenyl]amino}[1,2,4]triazolo[1,5-a]pyridin-7-yl)phenyl]propanamide

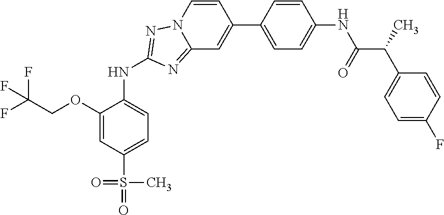 [SMILES: C[C@@H](C(=O)Nc1ccc(-c2ccn3nc(Nc4ccc(S(C)(=O)=O)cc4OCC(F)(F)F)nc3c2)cc1)c1ccc(F)cc1]

Starting from 7-chloro-N-[4-(methylsulfonyl)-2-(2,2,2-trifluoroethoxy)phenyl][1,2,4]triazolo[1,5-a]pyridin-2-amine (50 mg) and (4-{[(2R)-2-(4-fluorophenyl)propanoyl]amino}phenyl)boronic acid (51 mg), Reference Example 01.05. was prepared analogously to the procedure for the preparation of Reference Example 01.02. Yield: 20 mg of the title compound.

¹H-NMR (400 MHz, DMSO-d₆), δ [ppm]=1.42 (3H), 3.19 (3H), 3.87 (1H), 5.02 (2H), 7.12-7.20 (2H), 7.39-7.46 (3H), 7.62-7.67 (2H), 7.74 (2H), 7.81-7.88 (2H), 7.91 (1H), 8.53 (1H), 8.60 (1H), 8.85 (1H), 10.27 (1H).

### REFERENCE EXAMPLE 01.06

### (2R)—N-[4-(2-{[4-(azetidin-1-ylcarbonyl)-2-methoxyphenyl]amino}[1,2,4]triazolo[1,5-a]pyridin-7-yl)phenyl]-2-(4-fluorophenyl)propanamide

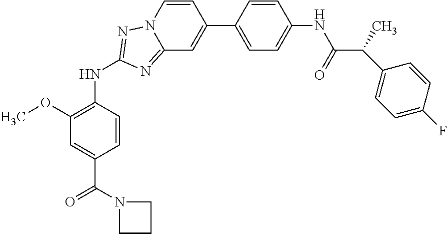 [SMILES: COc1cc(C(=O)N2CCC2)ccc1Nc1nc2cc(-c3ccc(NC(=O)[C@H](C)c4ccc(F)cc4)cc3)ccn2n1]

Starting from azetidin-1-yl{4-[(7-chloro[1,2,4]triazolo[1,5-a]pyridin-2-yl)amino]-3-methoxyphenyl}methanone (120 mg) and (4-{[(2R)-2-(4-fluorophenyl)propanoyl]amino}phenyl)boronic acid (144 mg), Reference Example 01.06. was prepared analogously to the procedure for the preparation of Reference Example 01.02. Yield: 30 mg of the title compound.

¹H-NMR (400 MHz, DMSO-d₆), δ [ppm]=1.42 (3H), 2.25 (2H), 3.82-3.94 (4H), 4.03 (2H), 4.36 (2H), 7.12-7.20 (2H), 7.22-7.29 (2H), 7.35-7.46 (3H), 7.73 (2H), 7.80-7.89 (3H), 8.29 (1H), 8.33 (1H), 8.81 (1H), 10.26 (1H).

### INTERMEDIATE EXAMPLE IntP01.01

Chloromethyl (7-chloro[1,2,4]triazolo[1,5-a]pyridin-2-yl){4-[(3-fluoroazetidin-1-yl)carbonyl]-2-(2,2,2-trifluoroethoxy)phenyl}carbamate

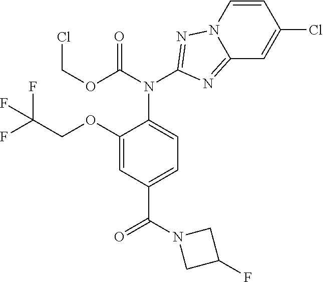 [SMILES: O=C(c1ccc(N(C(=O)OCCl)c2nc3cc(Cl)ccn3n2)c(OCC(F)(F)F)c1)N1CC(F)C1]

To a stirred solution of {4-[(7-chloro[1,2,4]triazolo[1,5-a]pyridin-2-yl)amino]-3-(2,2,2-trifluoroethoxy)phenyl}(3-fluoroazetidin-1-yl)methanone (120 mg) in THF (6 mL) and NMP (2.8 mL) was added sodium hydride (55% w/w in oil; 59 mg) at room temperature and the mixture was stirred for 15 minutes. Chloromethyl chloroformate (61 μL) was added at 0° C. and the mixture was stirred at room temperature for 1 hour. A half-saturated solution of ammonium chloride was added and the mixture was extracted with ethyl acetate. The organic phase was dried (sodium sulfate) and the solvent was removed in vacuum. Silicagel chromatography gave 75 mg of the title compound.

### INTERMEDIATE EXAMPLE IntP01.02

### caesium (2S)-2-[(tert-butoxycarbonyl)amino]-3,3-dimethylbutanoate

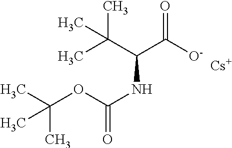

To a stirred solution of N-(tert-butoxycarbonyl)-3-methyl-L-valine (4.08 g) in methanol (36 mL) was added a solution of caesium carbonate in water until pH 7 was reached (approx. 2.85 g caesium carbonate in 36 mL water) and the solution was stirred for 30 minutes. The solvent was removed in vacuum, toluene was added and the solvent was again removed in vacuum to give 6.34 g of the title compound.

### INTERMEDIATE EXAMPLE IntP01.03

### {[(7-chloro[1,2,4]triazolo[1,5-a]pyridin-2-yl){4-[(3-fluoroazetidin-1-yl)carbonyl]-2-(2,2,2-trifluoroethoxy)phenyl}carbamoyl]oxy}methyl N-(tert-butoxycarbonyl)-3-methyl-L-valinate

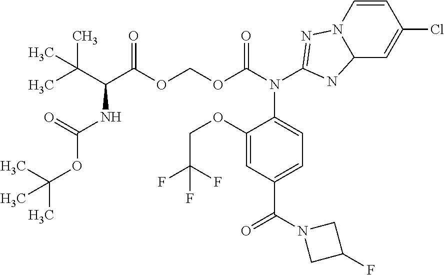 [SMILES: CC(C)(C)OC(=O)N[C@H](C(=O)OCOC(=O)N(C1=NN2C=CC(Cl)=CC2N1)c1ccc(C(=O)N2CC(F)C2)cc1OCC(F)(F)F)C(C)(C)C]

To a stirred solution of chloromethyl (7-chloro[1,2,4]triazolo[1,5-a]pyridin-2-yl){4-[(3-fluoroazetidin-1-yl)carbonyl]-2-(2,2,2-trifluoroethoxy)phenyl}carbamate (70 mg) in DMF (3.0 mL) was added caesium (2S)-2-[(tert-butoxycarbonyl)amino]-3,3-dimethylbutanoate (109 mg) and the mixture was stirred at room temperature for 72 h. A half-saturated solution of ammonium chloride was added and the mixture was extracted with ethyl acetate. The organic phase was washed with saturated sodium chloride solution, dried (sodium sulfate) and the solvent was removed in vacuum. Silicagel chromatography gave 68 mg of the title compound.

### INTERMEDIATE EXAMPLE IntP01.04

### [({4-[(3-fluoroazetidin-1-yl)carbonyl]-2-(2,2,2-trifluoroethoxy)phenyl}[7-(4-{[(2R)-2-(4-fluorophenyl)propanoyl]amino}phenyl)[1,2,4]triazolo[1,5-a]pyridin-2-yl]carbamoyl)oxy]methyl N-(tert-butoxycarbonyl)-3-methyl-L-valinate

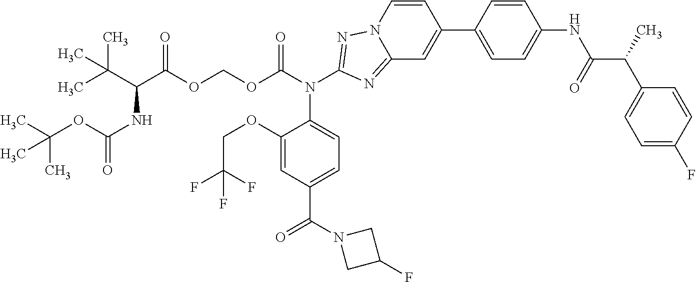 [SMILES: C[C@@H](C(=O)Nc1ccc(-c2ccn3nc(N(C(=O)OCOC(=O)[C@@H](NC(=O)OC(C)(C)C)C(C)(C)C)c4ccc(C(=O)N5CC(F)C5)cc4OCC(F)(F)F)nc3c2)cc1)c1ccc(F)cc1]

To a stirred solution of {[(7-chloro[1,2,4]triazolo[1,5-a]pyridin-2-yl){4-[(3-fluoroazetidin-1-yl)carbonyl]-2-(2,2,2-trifluoroethoxy)phenyl}carbamoyl]oxy}methyl N-(tert-butoxycarbonyl)-3-methyl-L-valinate (65 mg) in toluene (1.6 mL) and NMP (0.16 mL) was added (4-{[(2R)-2-(4-fluorophenyl)propanoyl]amino}phenyl)boronic acid (34.5 mg), powdered potassium phosphate monohydrate (68 mg), dicyclohexyl(2′,6′-dimethoxybiphenyl-2-yl)phosphine (6.6 mg) and palladium acetate (3.6 mg) and the flask was degassed twice and backfilled with argon. The mixture was heated to 100° C. for 20 minutes. The reaction mixture was filtered through a silica-gel column and the solvent was removed in vacuum to give a solid that was triturated with a mixture of hexane and dichloromethane to give 47 mg of the title compound.

Compounds of the present invention

### EXAMPLE 1.1

### [({4-[(3-fluoroazetidin-1-yl)carbonyl]-2-(2,2,2-trifluoroethoxy)phenyl}[7-(4-{[(2R)-2-(4-fluorophenyl)propanoyl]amino}phenyl)[1,2,4]triazolo[1,5-a]pyridin-2-yl]carbamoyl)oxy]methyl 3-methyl-L-valinate hydrochloride

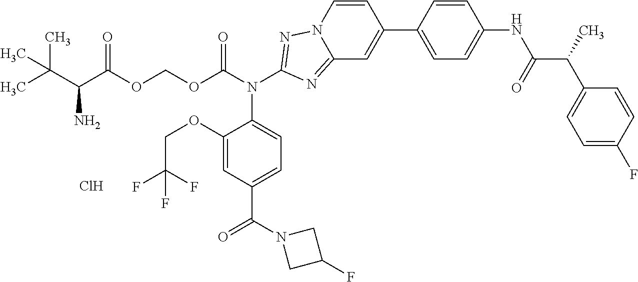 [SMILES: C[C@@H](C(=O)Nc1ccc(-c2ccn3nc(N(C(=O)OCOC(=O)[C@@H](N)C(C)(C)C)c4ccc(C(=O)N5CC(F)C5)cc4OCC(F)(F)F)nc3c2)cc1)c1ccc(F)cc1 ; Cl]

To a stirred solution of [({4-[(3-fluoroazetidin-1-yl)carbonyl]-2-(2,2,2-trifluoroethoxy)phenyl}[7-(4-{[(2R)-2-(4-fluorophenyl)propanoyl]annino}phenyl)[1,2,4]triazolo[1,5-a]pyridin-2-yl]carbamoyl)oxy]methyl N-(tert-butoxycarbonyl)-3-methyl-L-valinate (44 mg) in dichloromethane (1 mL) and methanol (0.3 mL) was added a solution of hydrochloric acid in dioxane (0.24 mL; c=4.0 M). The mixture was stirred at room temperature for 2 h. The solvent was removed in vacuum. The solid residue was triturated with a mixture of dichloromethane and hexane for three times, the solvent was removed each time and the solid was dried in vacuum to give 32 mg of the title compound.

¹H-NMR (400 MHz, DMSO-d₆): δ [ppm]=0.94-1.01 (m, 9H), 1.41 (d, 3H), 3.82-3.96 (m, 2H), 3.99-4.19 (m, 1H), 4.31-4.69 (m, 3H), 4.83 (q, 2H), 5.33-5.56 (m, 1H), 5.85 (d, 1H), 5.95 (d, 1H), 7.11-7.19 (m, 2H), 7.33-7.47 (m, 5H), 7.52 (dd, 1H), 7.71-7.85 (m, 4H), 7.97 (d, 1H), 8.44 (d, 3H), 8.86 (d, 1H), 10.38 (s, 1H).

LC-MS (Method 2): Rₜ=1.15 min; MS (ESIpos) m/z=838 [M+H]⁺.

Biological Assay: Proliferation Assay

Cultivated tumor cells (MCF7, hormone dependent human mammary carcinoma cells, ATCC HTB22; NCI-H460, human non-small cell lung carcinoma cells, ATCC HTB-177; DU 145, hormone-independent human prostate carcinoma cells, ATCC HTB-81; HeLa-MaTu, human cervical carcinoma cells, EPO-GmbH, Berlin; HeLa-MaTu-ADR, multidrug-resistant human cervical carcinoma cells, EPO-GmbH, Berlin; HeLa human cervical tumor cells, ATCC CCL-2; B16F10 mouse melanoma cells, ATCC CRL-6475) were plated at a density of 5000 cells/well (MCF7, DU145, HeLa-MaTu-ADR), 3000 cells/well (NCI-H460, HeLa-MaTu, HeLa), or 1000 cells/well (B16F10) in a 96-well multititer plate in 200 μl of their respective growth medium supplemented 10% fetal calf serum. After 24 hours, the cells of one plate (zero-point plate) were stained with crystal violet (see below), while the medium of the other plates was replaced by fresh culture medium (200 μl), to which the test substances were added in various concentrations (0 μM, as well as in the range of 0.01-30 μM; the final concentration of the solvent dimethyl sulfoxide was 0.5%). The cells were incubated for 4 days in the presence of test substances. Cell proliferation was determined by staining the cells with crystal violet: the cells were fixed by adding 20 μl/measuring point of an 11% glutaric aldehyde solution for 15 minutes at room temperature. After three washing cycles of the fixed cells with water, the plates were dried at room temperature. The cells were stained by adding 100 μl/measuring point of a 0.1% crystal violet solution (pH 3.0). After three washing cycles of the stained cells with water, the plates were dried at room temperature. The dye was dissolved by adding 100 μl/measuring point of a 10% acetic acid solution. The extinction was determined by photometry at a wavelength of 595 nm. The change of cell number, in percent, was calculated by normalization of the measured values to the extinction values of the zero-point plate (=0%) and the extinction of the untreated (0 μm) cells (=100%). The IC₅₀ values were determined by means of a 4 parameter fit.

The reference examples described above are characterized by the following IC₅₀ values, determined in a HeLa cell proliferation assay (as described above):

| | Inhibition of cell |
|---|---|
| | proliferation, |
| Reference | cell Line: HeLa |
| Example | IC₅₀ |
| 01.01. | ≦200 nM |
| 01.02. | ≦100 nM |
| 01.03. | ≦100 nM |
| 01.05. | ≦100 nM |
| 01.06. | ≦100 nM |

Mps-1 Kinase Assay

The human kinase Mps-1 phosphorylates a biotinylated substrate peptide. Detection of the phosphorylated product is achieved by time-resolved fluorescence resonance energy transfer (TR-FRET) from Europium-labelled anti-phospho-Serine/Threonine antibody as donor to streptavidin labelled with cross-linked allophycocyanin (SA-XLent) as acceptor. Compounds are tested for their inhibition of the kinase activity.

N-terminally GST-tagged human full length recombinant Mps-1 kinase (purchased from Invitrogen, Karslruhe, Germany, cat. no PV4071) was used. As substrate for the kinase reaction a biotinylated peptide of the amino-acid sequence PWDPDDADITEILG (C-terminus in amide form, purchased from Biosynthan GmbH, Berlin) was used.

For the assay 50 nl of a 100-fold concentrated solution of the test compound in DMSO was pipetted into a black low volume 384well microtiter plate (Greiner Bio-One, Frickenhausen, Germany), 2 μl of a solution of Mps-1 in assay buffer [0.1 mM sodium-ortho-vanadate, 10 mM MgCl₂, 2 mM DTT, 25 mM Hepes pH 7.7, 0.05% BSA, 0.001% Pluronic F-127] were added and the mixture was incubated for 15 min at 22° C. to allow pre-binding of the test compounds to Mps-1 before the start of the kinase reaction. Then the kinase reaction was started by the addition of 3 μl of a solution of 16.7 adenosine-tri-phosphate (ATP, 16.7 μM=>final conc. in the 5 μl assay volume is 10 μM) and peptide substrate (1.67 μM=>final conc. in the 5 μl assay volume is 1 μM) in assay buffer and the resulting mixture was incubated for a reaction time of 60 min at 22° C. The concentration of Mps-1 in the assay was adjusted to the activity of the enzyme lot and was chosen appropriate to have the assay in the linear range, typical enzyme concentrations were in the range of about 1 nM (final conc. in the 5 μl assay volume). The reaction was stopped by the addition of 3 μl of a solution of HTRF detection reagents (100 mM Hepes pH 7.4, 0.1% BSA, 40 mM EDTA, 140 nM Streptavidin-XLent [#61GSTXLB, Fa. Cis Biointernational, Marcoule, France], 1.5 nM anti-phospho(Ser/Thr)-Europium-antibody [#AD0180, PerkinElmer LAS, Rodgau-Jugesheinn, Germany].

The resulting mixture was incubated 1 h at 22° C. to allow the binding of the phosphorylated peptide to the anti-phospho(Ser/Thr)-Europium-antibody. Subsequently the amount of phosphorylated substrate was evaluated by measurement of the resonance energy transfer from the Europium-labelled anti-phospho(Ser/Thr) antibody to the Streptavidin-XLent. Therefore, the fluorescence emissions at 620 nm and 665 nm after excitation at 350 nm was measured in a Viewlux TR-FRET reader (PerkinElmer LAS, Rodgau-Jugesheinn, Germany). The “blank-corrected normalized ratio” (a Viewlux specific readout, similar to the traditional ratio of the emissions at 665 nm and at 622 nm, in which blank and Eu-donor crosstalk are subtracted from the 665 nm signal before the ratio is calculated) was taken as the measure for the amount of phosphorylated substrate. The data were normalised (enzyme reaction without inhibitor=0% inhibition, all other assay components but no enzyme=100% inhibition). Test compounds were tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100fold conc. stock solutions by serial 1:3 dilutions) in duplicate values for each concentration and IC₅₀ values were calculated by a 4 parameter fit.

The reference examples described above are characterized by the following IC₅₀ values, determined in Mps-1 kinase assays (as described above):

| | Mps-1 Inhibition, IC₅₀ |
|---|---|
| Reference | (Assay with 10 μM |
| Example | ATP) |
| 01.01. | ≦1 nM |
| 01.02. | ≦1 nM |
| 01.03. | ≦1 nM |
| 01.05. | ≦1 nM |
| 01.06. | ≦1 nM |

Spindle Assembly Checkpoint Assay

The spindle assembly checkpoint assures the proper segregation of chromosomes during mitosis. Upon entry into mitosis, chromosomes begin to condensate which is accompanied by the phosphorylation of histone H3 on serine 10. Dephosphorylation of histone H3 on serine 10 begins in anaphase and ends at early telophase. Accordingly, phosphorylation of histone H3 on serine 10 can be utilized as a marker of cells in mitosis. Nocodazole is a microtubule destabilizing substance. Thus, nocodazole interferes with microtubule dynamics and mobilises the spindle assembly checkpoint. The cells arrest in mitosis at G2/M transition and exhibit phosphorylated histone H3 on serine 10. An inhibition of the spindle assembly checkpoint by Mps-1 inhibitors overrides the mitotic blockage in the presence of nocodazole, and the cells complete mitosis prematurely. This alteration is detected by the decrease of cells with phosphorylation of histone H3 on serine 10. This decline is used as a marker to determine the capability of compounds of the present invention to induce a mitotic breakthrough.

Cultivated cells of the human cervical tumor cell line HeLa (ATCC CCL-2) were plated at a density of 2500 cells/well in a 384-well microtiter plate in 20 μl Dulbeco's Medium (w/o phenol red, w/o sodium pyruvate, w 1000 mg/ml glucose, w pyridoxine) supplemented with 1% (v/v) glutamine, 1% (v/v) penicillin, 1% (v/v) streptomycin and 10% (v/v) fetal calf serum. After incubation overnight at 37° C., 10 μl/well nocodazole at a final concentration of 0.1 μg/ml were added to cells. After 24 h incubation, cells were arrested at G2/M phase of the cell cycle progression. Test compounds solubilised in dimethyl sulfoxide (DMSO) were added at various concentrations (0 μM, as well as in the range of 0.005 μM-10 μM; the final concentration of the solvent DMSO was 0.5% (v/v)). Cells were incubated for 4 h at 37° C. in the presence of test compounds. Thereafter, cells were fixed in 4% (v/v) paraformaldehyde in phosphate buffered saline (PBS) at 4° C. overnight then permeabilised in 0.1% (v/v) Triton X™ 100 in PBS at room temperature for 20 min and blocked in 0.5% (v/v) bovine serum albumin (BSA) in PBS at room temperature for 15 min. After washing with PBS, 20 μl/well antibody solution (anti-phospho-histone H3 clone 3H10, FITC; Upstate, Cat# 16-222; 1:200 dilution) was added to cells, which were incubated for 2 h at room temperature. Afterwards, cells were washed with PBS and 20 μl/well HOECHST 33342 dye solution (5 μg/ml) was added to cells and cells were incubated 12 min at room temperature in the dark. Cells were washed twice with PBS then covered with PBS and stored at 4° C. until analysis. Images were acquired with a Perkin Elmer OPERA™ High-Content Analysis reader. Images were analyzed with image analysis software MetaXpress™ from Molecular devices utilizing the Cell Cycle application module. In this assay both labels HOECHST 33342 and phosphorylated Histone H3 on serine 10 were measured. HOECHST 33342 labels DNA and is used to count cell number. The staining of phosphorylated Histone H3 on serine 10 determines the number of mitotic cells. Inhibition of Mps-1 decreases the number of mitotic cells in the presence of nocodazole indicating an inappropriate mitotic progression. The raw assay data were further analysed by four parameter logistic regression analysis to determine the IC₅₀ value for each tested compound.

Stability in Buffer at pH 7.4

0.3 mg of the test compound are solved in 0.1 ml dimethylsulfoxide and 0.4 ml acetonitrile. For complete dissolution the HPLC vial with the sample solution is sonified for about 20 seconds. Then 1.0 ml of the buffer solution is added, and the sample is again treated in the ultrasonic bath.

Preparation of the buffer solution:

90 g of sodium chloride, 13.61 g of potassium dihydrogen phosphate and 83.35 g of 1 M sodium hydroxide solution are made up to 1 litre with Millipore water and then diluted 1:10.

10 μl portions of the sample solution are analysed by HPLC to determine the amount of the test compound over a period of 24 hours at 37° C. The peak areas in percentage are used for quantification.

HPLC Method:

Agilent 1100 with DAD (G1315B), binary pump (G1312A), autosampler (G1329A), column oven (G1316A), thermostat (G1330B); column: Kromasil 100 C18, 250 mm×4 mm, 5 μm; column temperature: 37° C.; eluent A: water+5 ml of perchloric acid/litre, eluent B: acetonitrile.

Gradient:

0 min 98% A, 2% B→0-3.0 min 85% A, 15% B→3.0-8.0 min 50% A, 50% B→8.0-16.0 min 50% A, 50% B→16.0-20.0 min 10% A, 90% B→20.0-21.0 10% A, 90% B→21.0-24.0 min 98% A, 2% B→24.0-25.0 min 98% A, 2% B; flow rate: 1.5 ml/min; UV detection: 210 nm.

The ratio of the peak areas (F) at different time points in relation to the peak areas at the starting time are shown in Table 1 for representative examples:

TABLE 1
Stability in buffer at pH 7.4

| | % Test Compound |
|---|---|
| | after 24 h |
| Example No. | [F(t = 24 h) × 100/F(t = 0 h)] |
| 1.1. | 0.0 |

In vitro stability in rat and human plasma (HPLC detection)

1 mg of test compound is dissolved in 1.25 ml dimethylsulfoxide. Then 1.25 ml water are added. 0.5 ml of this sample solution are mixed with 0.5 ml heparinized and 37° C. warm plasma (wistar rat plasma or human plasma). A first sample (10 μl) is immediately taken for HPLC analysis. In the period up to 4 h after the start of incubation further 10 μl aliquots are taken after 30, 60, 90, 120 and 240 minutes and the amount of the test compound is determined.

HPLC method:

Agilent 1100 with DAD (G1315A), binary pump (G1312A), autosampler (G1329A), column oven (G1316A), thermostat (G1330B); column: Kromasil 100 C18, 250 mm×4 mm, 5 μm; column temperature: 45° C.; eluent A: water+5 ml of perchloric acid/litre, eluent B: acetonitrile.

Gradient:

0 min 98% A, 2% B→0-3.0 min 85% A, 15% B→3.0-8.0 min 55% A, 45% B→8.0-16.0 min 55% A, 45% B→16.0-20.0 min 10% A, 90% B→20.0-21.0 10% A, 90% B→21.0-24.0 min 98% A, 2% B→24.0-25.0 min 98% A, 2% B; flow rate: 1.5 ml/min; UV detection: 222 nm.

The ratio of the peak areas (F) at the respective time points in relation to the peak areas at the starting time indicates the remaining parent compount, hence indicating stability under the experimental conditions described.

TABLE 2
In vitro stability in rat plasma

| | % Test Compound |
|---|---|
| | after 4 h |
| Example No. | [F(t = 24 h) × 100/F(t = 0 h)] |
| 1.1. | 0.0 |

TABLE 3
In vitro stability in human plasma

| | % Test Compound |
|---|---|
| | after 4 h |
| Example No. | [F(t = 24 h) × 100/F(t = 0 h)] |
| 1.1. | 0.0 |

Determination of Metabolic Stability In Vitro

(including calculation of hepatic in vivo blood clearance (CL) and of maximal oral bioavailability (Fₘₐₓ))

The metabolic stability of test compounds in vitro was determined by incubating them at 1 μM with a suspension liver microsomes in 100 mM phosphate buffer, pH7.4 (NaH₂PO₄×H₂O+Na₂HPO₄×2H₂O) at a protein concentration of 0.5 mg/mL and at 37° C. The reaction was activated by adding a co-factor mix containing 1.2 mg NADP, 3 IU glucose-6-phosphate dehydrogenase, 14.6 mg glucose-6-phosphate and 4.9 mg MgCl₂ in phosphate buffer, pH 7.4. Organic solvent in the incubations was limited to <0.2% dimethylsulfoxide (DMSO) and <1% methanol. During incubation, the microsomal suspensions were continuously shaken and aliquots were taken at 2, 8, 16, 30, 45 and 60 min, to which equal volumes of cold methanol were immediately added. Samples were frozen at −20° C. over night, subsequently centrifuged for 15 minutes at 3000 rpm and the supernatant was analyzed with an Agilent 1200 HPLC-system with LCMS/MS detection.

The half-life of a test compound was determined from the concentration-time plot. From the half-life the intrinsic clearances were calculated. Together with the additional parameters liver blood flow, specific liver weight and microsomal protein content the hepatic in vivo blood clearance (CL) and the maximal oral bioavailability (Fₘₐₓ) were calculated for the different species. The following parameter values were used: Liver blood flow −1.3 L/h/kg (human), 2.1 L/h/kg (dog), 4.2 L/h/kg (rat); specific liver weight −21 g/kg (human), 39 g/kg (dog), 32 g/kg (rat); microsomal protein content −40 mg/g.

With the described assay only phase-I metabolism of microsomes is reflected, e.g. typically oxidoreductive reactions by cytochrome P450 enzymes and flavin mono-oxygenases (FMO) and hydrolytic reactions by esterases (esters and amides).

1. A compound of general formula (I): 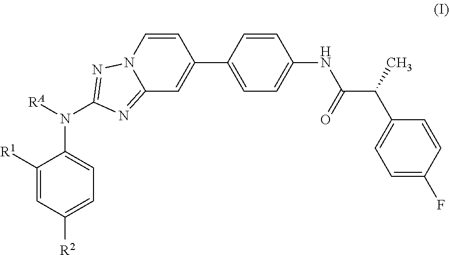
in which:
R^{A} represents —C(═O)—O—C(R⁴)(R⁵)—O—C(═O)—C(R³)(NH₂)—R⁶;
R¹ represents a group selected from the group consisting of methoxy- and 2,2,2-trifluoroethoxy-;
R² represents a group selected from the group consisting of: 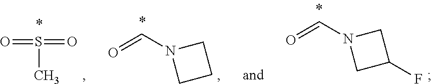
wherein “*” indicates the point of attachment to the phenyl ring R² is attached to;
R³ represents a hydrogen atom or a methyl-group;
R⁴ and R⁵, independently from each other, represent a hydrogen atom or a C₁-C₃-alkyl-group; and
R⁶ represents a hydrogen atom or a C₁-C₆-alkyl-group;
or an N-oxide, a hydrate, a solvate, or a salt thereof, or a mixture of same.

2. The compound according to claim 1, wherein:
R^{A} represents a group selected from the group consisting of:
R⁶—C(R³)(NH₂)—C(═O)—O—CH₂—O—C(═O)—,
R⁶—C(R³)(NH₂)—C(═O)—O—C(H)(CH₃)—O—C(═O)—, and
R⁶—C(R³)(NH₂)—C(═O)—O—C(H)(C(H)(CH₃)₂)—O—C(═O)—;
or an N-oxide, a hydrate, a solvate, or a salt thereof, or a mixture of same.

3. The compound according to claim 1, wherein:
R^{A} represents a group selected from the group consisting of: 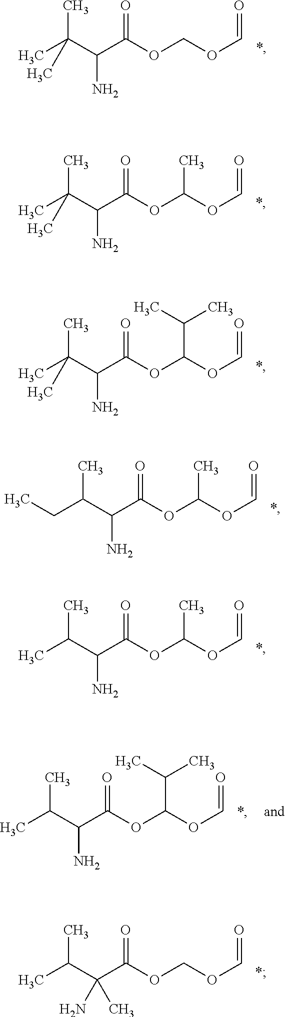
wherein “*” indicates the point of attachment to the nitrogen atom R^{A} is attached to:
or an N-oxide, a hydrate, a solvate, or a salt thereof, or a mixture of same.

4. The compound according to claim 1 wherein:
R^{A} represents a group selected from the group consisting of: 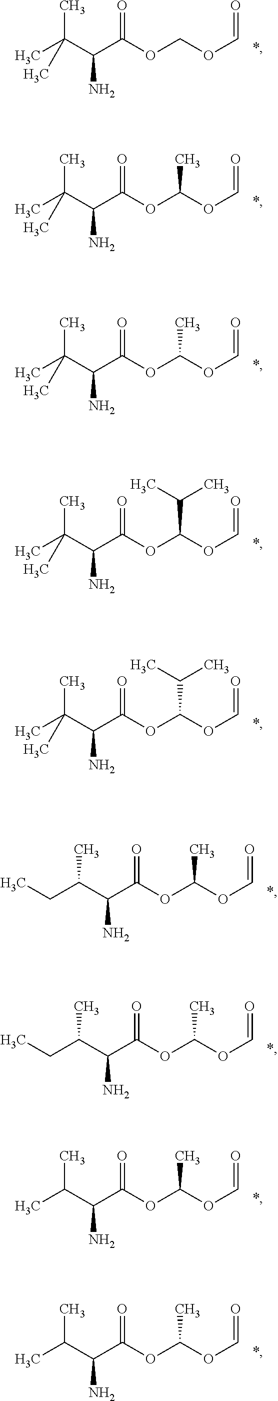 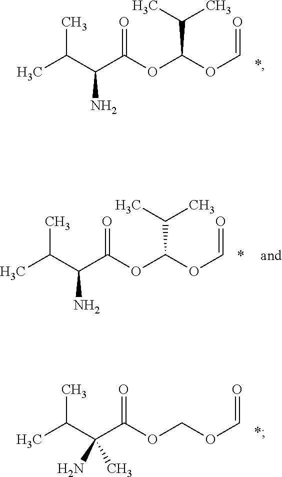
wherein “*” indicates the point of attachment to the nitrogen atom R^{A} is attached to:
or an N-oxide, a hydrate, a solvate, or a salt thereof, or a mixture of same.

5. The compound according to claim 1, wherein the compound of general formula (I) is a compound of general formula (Ia), a compound of general formula (Ib), a compound of general formula (Ic), a compound of general formula (Id), a compound of general formula (Ie) or a compound of general formula (If): 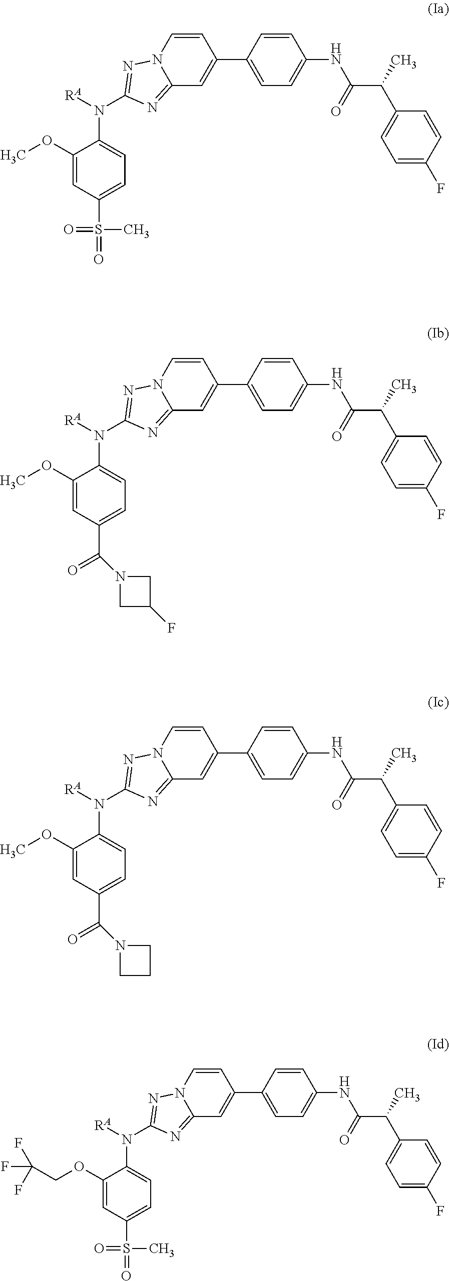 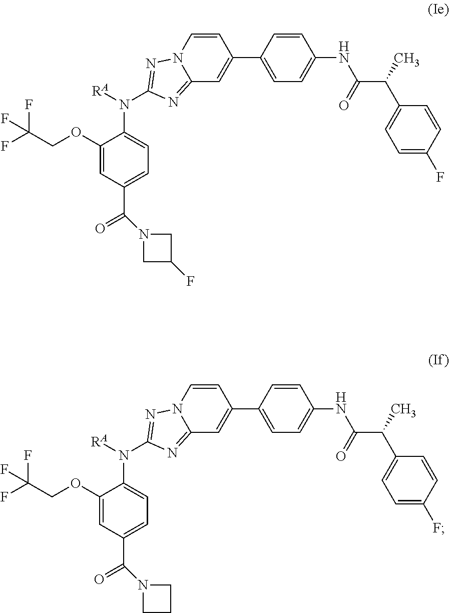
or an N-oxide, a hydrate, a solvate, or a salt thereof, or a mixture of same.

6. The compound according to claim 1, which is [({4-[(3-fluoroazetidin-1-yl)carbonyl]-2-(2,2,2-trifluoroethoxy)phenyl}[7-(4-{[(2R)-2-(4-fluorophenyl)propanoyl]amino}phenyl)[1,2,4]triazolo[1,5-a]pyridin-2-yl]carbamoyl)oxy]methyl 3-methyl-L-valinate;
or an N-oxide, a hydrate, a solvate, or a salt thereof, or a mixture of same.

7. (canceled)

8. A pharmaceutical composition comprising a compound according to claim 1, or an N-oxide, a hydrate, a solvate, or a salt thereof, or a mixture of same, and a pharmaceutically acceptable diluent or carrier.

9-10. (canceled)

11. A method for the prophylaxis or treatment of a disease, comprising administering to a patient in need thereof a pharmaceutically effective amount of a compound according to claim 1, or an N-oxide, a hydrate, a solvate, or a salt thereof, or a mixture of same; wherein said disease is a disease of uncontrolled cell growth, proliferation or survival, an inappropriate cellular immune response, or an inappropriate cellular inflammatory response.

12. The method according to claim 11, wherein the uncontrolled cell growth, proliferation or survival, inappropriate cellular immune response, or inappropriate cellular inflammatory response is mediated by Mps-1.

13. The method according to claim 11, wherein the uncontrolled cell growth, proliferation or survival, inappropriate cellular immune response, or inappropriate cellular inflammatory response is a haemotological tumour, a solid tumour, or a metastase thereof.

14. The method according to claim 11, wherein the uncontrolled cell growth, proliferation or survival, inappropriate cellular immune response, or inappropriate cellular inflammatory response is a leukaemia, myelodysplastic syndrome, a malignant lymphoma, a head and neck tumour, a tumour of the thorax, a gastrointestinal tumour, an endocrine tumour, a mammary tumour, a gynaecological tumour, a urological tumour, a bladder and prostate tumour, a skin tumour, a sarcoma, or a metastase thereof.

15. The method according to claim 14, wherein the uncontrolled cell growth, proliferation or survival, inappropriate cellular immune response, or inappropriate cellular inflammatory response is a head and neck tumour selected from the group consisting of a brain tumour and a brain metastase.

16. The method according to claim 14, wherein the uncontrolled cell growth, proliferation or survival, inappropriate cellular immune response, or inappropriate cellular inflammatory response is a tumour of the thorax selected from the group consisting of a non-small cell lung tumour and a small cell lung tumour.

17. The method according to claim 14, wherein the uncontrolled cell growth, proliferation or survival, inappropriate cellular immune response, or inappropriate cellular inflammatory response is a urological tumour, wherein the urological tumour is a renal tumour.

18. A pharmaceutical composition comprising a compound according to claim 6, or an N-oxide, a hydrate, a solvate, or a salt thereof, or a mixture of same, and a pharmaceutically acceptable diluent or carrier.

19. A method for the prophylaxis or treatment of a disease, comprising administering to a patient in need thereof a pharmaceutically effective amount of a compound according to claim 6, or an N-oxide, a hydrate, a solvate, or a salt thereof, or a mixture of same; wherein said disease is a disease of uncontrolled cell growth, proliferation or survival, an inappropriate cellular immune response, or an inappropriate cellular inflammatory response.

20. The method according to claim 19, wherein the uncontrolled cell growth, proliferation or survival, inappropriate cellular immune response, or inappropriate cellular inflammatory response is mediated by Mps-1.

21. The method according to claim 19, wherein the uncontrolled cell growth, proliferation or survival, inappropriate cellular immune response, or inappropriate cellular inflammatory response is a haemotological tumour, a solid tumour, or a metastase thereof.

22. The method according to claim 19, wherein the uncontrolled cell growth, proliferation or survival, inappropriate cellular immune response, or inappropriate cellular inflammatory response is a leukaemia, myelodysplastic syndrome, a malignant lymphoma, a head and neck tumour, a tumour of the thorax, a gastrointestinal tumour, an endocrine tumour, a mammary tumour, a gynaecological tumour, a urological tumour, a bladder and prostate tumour, a skin tumour, a sarcoma, or a metastase thereof.
